(19) United States
(12) Patent Application Publication
(10) Pub. No.: US 2010/0322945 A1
(43) Pub. Date: Dec. 23, 2010

(54) IMMUNOGENIC COMPOUNDS AND PROTEIN MIMICS

(76) Inventors: Peter Timmerman, Lelystad (NL); Wouter Cornelis Puijk, Lelystad (NL); Robbert Hans Meloen, Lelystad (NL)

Correspondence Address: TRASKBRITT, P.C., P.O. BOX 2550, SALT LAKE CITY, UT, 84110, US

(21) Appl. No.: 12/309,751

(22) PCT Filed: Jul. 26, 2007
(86) PCT No.: PCT/NL2007/050374

§ 371 Date: Jul. 7, 2009

(30) Foreign Application Priority Data
Jul. 26, 2006 (EP) 06076478.4

(51) Int. Cl. A61K 39/385; C07K 1/00; C07K 17/02; A61K 39/395; A61P 35/00

(52) U.S. Cl. 424/152.1; 424/193.1; 530/324; 530/325; 530/326; 530/327; 530/328; 530/329; 530/330; 530/331; 424/195.11; 424/172.1

(57) ABSTRACT

The invention provides means and methods for inducing and/or enhancing immunogenic reproducibility and/or immunogenicity of a compound, comprising at least in part restricting the conformation of said compound. The conformation is preferably restricted by the formation of at least one internal bond within said compound. Protein mimics comprising an amino acid sequence bound to a scaffold and/or a carrier, wherein said amino acid sequence comprises at least one internal SS-bridge, are also herewith provided.

The invention relates to the fields of biology and immunology.

There is an ever expanding interest in the art in detection, identification, isolation and generation of immunogenic compounds. Immunogenic compounds are used in a wide variety of applications, such as for instance vaccination programs and the production of antibodies, B cells and T cells of interest. During vaccination an immunogenic compound is used in order to evoke an immune response in a host, which immune response is preferably a protective immune response against a disease related to the presence of an antigen of interest such as for instance a pathogen or a tumor. Such immunogenic compounds typically comprise a peptide sequence that is wholly or in part derived from said antigen of interest.

Immunogenic compounds are also widely used for obtaining antibodies, B cells and/or T cells of interest. Non-human animals are immunized with an immunogenic compound, where after antibodies, B cells and/or T cells are harvested from the animal for further use. Particularly, the production of monoclonal antibodies (mAbs) starting from an immunogenic compound of interest is an important application. Amongst the benefits of mAbs is their ability to target specific cells or chemical mediators that could be involved in disease causation. This specificity confers certain clinical advantages on mAbs over more conventional treatments while offering patients an effective, well-tolerated therapy option with generally low side effects.

Despite many successful developments in the art, immunization not always yields the desired effect. For instance, immunization against self-antigens is difficult because a host's immune system is in principle not directed against self-antigens. An immune response against a self-antigen is however desired in various cases, for instance when a self-antigen is present on tumor cells. An immune response against a self-antigen present on a tumor would counteract the tumor. Moreover, tumor growth requires angiogenesis, which involves the formation of new blood vessels, in order to carry nutrients to the site of the tumor and to transport waste material from the tumor. Angiogenesis involves the action of endogenous growth hormones such as ’ vascular endothelial growth factor (VEGF), also called vascular permeability factor (VPF). Hence, counteracting the action of such growth factor would indirectly counteract tumor growth because angiogenesis would be hampered.

In view of the fact that a host's immune response is in general inactive against self antigens, immunogenic compounds are often generated which are sufficient different from the self antigen in order to elicit an immune response, yet sufficiently resemble the self antigen such that an elicited immune response is also active against the self antigen. Furthermore, immunogenicity is often enhanced with a carrier, such as for instance keyhole limpet hemocyanin (KLH), and/or an adjuvant such as for instance (incomplete) Freund's adjuvant. However, a sufficiently strong immune response against an antigen of interest is still not always obtained.

Another problem encountered in the field of immunology is lack of immunogenic reproducibility. This means that a desired immune response is obtained in one animal, but not, or to a significantly lesser extent, in a second animal of the same species even though both animals are immunized with the same kind of immunogenic compound. This variability in immune responses between animals of the same species is not well understood. Biologic diversity between individuals is generally considered to result in differences between immune systems of different animals.

It is an object of the present invention to provide means and methods for improving the immunogenicity of a compound of interest. It is a further object to induce and/or enhance immunogenic reproducibility.

The invention in one aspect provides a method for inducing and/or enhancing immunogenic reproducibility and/or immunogenicity of a compound, the method comprising at least in part restricting the conformation of said compound.

According to the present invention, immunogenicity of a compound of interest is induced and/or improved by (further) restricting the conformation of said compound. Immunogenicity is even improved when said compound already has a rather limited conformation, for instance due to a linkage to a carrier. Even then, further restricting the conformation significantly improves immunogenicity. If a compound with (further) restricted conformation is administered to an animal, an immune response against a certain antigen of interest is elicited which is stronger as compared to the situation wherein the same kind of compound with a less restricted conformation is administered to the same kind of animal. The extent of an immune response is for instance determined by measuring antibody titer in a blood sample of an immunized animal.

Moreover, according to the invention immunogenic reproducibility of an immunogenic compound is induced and/or enhanced by (further) restricting the conformation of said compound. With a method of the invention it has become possible to generate immunogenic compounds which, when administered to a group of animals of the same species, are capable of inducing the same kind of immune response in a larger part of said group of animals as compared to current immunogenic compounds. Hence, an immunogenic compound according to the invention with restricted conformation is capable of eliciting a comparable immune response in a higher percentage of a group of animals of the same species as compared to the same kind of compound with a less restricted conformation. Animal to animal variation is reduced. This means that, if several animals of the same species are immunized with an immunogenic compound according to the invention, the extent of the elicited immune responses of the animals will differ to a lower extent as compared to animals of the same species immunized with current immunogenic compounds. Hence, the spread is reduced. Moreover, it has become possible to induce an immune response in a larger part of a population. The chance of obtaining a significant immune response is therefore increased for each individual. This is for instance particularly advantageous when a group of animals, or a human population, is vaccinated (because protection of each individual against disease is desired), when several non-human animals are immunized in order to obtain antibodies, T cells and/or B cells (in order to obtain maximum yield), and during medical research (because differences in immunogenic test results due to animal to animal variations are reduced).

Any animal capable of eliciting an immune response against an immunogenic compound is encompassed by the term “animal”. An animal preferably comprises a mammal. In one preferred aspect said animal comprises a human individual. However, in other embodiments said animal comprises a non-human animal, for instance when an immune response is induced in order to harvest antibodies, T cells and/or B cells.

The conformation of a compound is defined as the number of possible spatial arrangements of a compound. In view of rotation about single covalent bonds, free compounds often adopt many different conformations. Restricting the conformation of a compound involves limiting the number of possible spatial arrangements, thereby forcing the compound to spend more time in a certain conformational state.

Immunogenic reproducibility is defined herein as the chance that the same kind of immune response which is obtained in one animal is also obtained in a second animal of the same species when both animals are immunized with the same immunogenic compound. If immunogenic reproducibility is enhanced, a larger percentage of a group of animals will exhibit the same kind of immune response. By the same kind of immune response is meant that the specificity and extent of the immune responses are comparable. If antibody titer is measured, meaning the antibody concentration in serum, often given as the value of serum dilution at which the OD in a binding ELISA is >3× the background-OD, an immune response in a first animal is comparable to an immune response in a second animal if the antibody titers of both animal differ less than hundredfold, preferably less than fifty fold, most preferably less than thirty fold. If antibody titers are given as Log values, an immune response in a first animal is comparable to an immune response in a second animal if the antibody titers of both animals differ less than 2.0, preferably less than 1.5.

Immunogenicity of a compound is defined as the capability of a compound of eliciting an immune response specifically directed against the compound itself and/or, preferably, against a molecule of interest. Said molecule of, interest preferably comprises a proteinaceous molecule. Preferably, said compound is capable of eliciting antibodies which strongly bind to, and neutralize the biological activity of, said molecule of interest.

A proteinaceous molecule is defined as a molecule comprising amino acid residues bound to each other via a peptide bond. Said molecule may comprise one or several non-amino acid moieties.

A compound's capability of eliciting an immune response specifically directed against a (proteinaceous) molecule of interest is called cross-reactivity. A method according to the invention is preferably used for inducing and/or enhancing cross-reactivity of a compound. A preferred embodiment therefore provides a method for inducing and/or enhancing cross-reactivity of a compound, the method comprising at least in part restricting the conformation of said compound. An immunogenic compound is preferably derived from a proteinaceous molecule of interest, meaning that said immunogenic compound comprises an amino acid sequence which is at least 50% homologous to said proteinaceous molecule of interest. An immunogenic compound comprising an amino acid sequence which is at least 50% homologous to a proteinaceous molecule of interest is preferably capable of inducing an immune response which is specific for said proteinaceous molecule of interest. An immunogenic compound according to the invention thus preferably comprises an amino acid sequence comprising a sequence which is at least 50% homologous to at least part of the amino acid sequence of a proteinaceous molecule of interest, said part having a length of at least 8 amino acid residues. In a preferred embodiment said amino acid sequence comprises a sequence which is at least 60%, preferably at least 70%, more preferably at least 80%, more preferably at least 90%, more preferably at least 95%, most preferably at least 97% homologous to at least part of the amino acid sequence of a proteinaceous molecule of interest, said part having a length of at least 8 amino acid residues. Such immunogenic compound is particularly suitable for eliciting an immune response against a proteinaceous molecule of interest. In one preferred embodiment said immunogenic compound is capable of eliciting a stronger immune response against said protein aceous molecule of interest as compared to a situation wherein an animal is immunized with said proteinaceous molecule itself. This is for instance possible by modifying an amino acid sequence derived from a self-antigen. Since an individual's immune system is in principle not active towards self-antigens, a modified sequence is often better capable of eliciting an immune response as compared to the native sequence. Methods for improving immunogenicity of an amino acid sequence for instance comprise a TDK-Alascan method and/or replacement net mapping method, which are well known in the art. TDK-Alascan involves substitution of an original amino acid residue by alanine. In a replacement net mapping method an original amino acid residue is replaced by any other amino acid residue. Preferably, a plurality of molecules is generated, wherein different amino acid residues are replaced, either by alanine or by any other amino acid residue. Subsequently immunogenicity (preferably comprising cross-reactivity) of the resulting molecules is tested, for instance by determining binding affinity to an antibody capable of specifically binding an antigen of interest. A molecule with a desired characteristic is subsequently identified and/or isolated. Said molecule is either used for immunization, or further optimized in another round of substitution and selection method. Of course, other optimization procedures are applicable as well.

In one embodiment an amino acid sequence is used that is at least 50%, preferably at least 60%, more preferably at least 70, 80, 90 or 95%, homologous to a non immunodominant site of a proteinaceous molecule of interest. Immunodominant sites are sites against which an immune response is primarily directed after immunization with a proteinaceous molecule of interest. Such immunodominant sites are for instance easily accessible. However, it is often desired to elicit antibodies against another specific site which is not immunodominant, such as for instance a receptor binding site. In that case the use of a peptide derived from said specific non-immunodominant site is preferred. This embodiment is for instance particularly suitable for inducing and/or enhancing an immune response against a receptor binding site of G Protein-Coupled Receptors (GPCR's), such as for instance the chemokine receptors CCR5 and/or CXCR4.

One aspect of the invention thus provides a method according to the invention wherein an immunogenic compound with restricted conformation is capable of inducing and/or enhancing an immune response in an animal against a proteinaceous molecule of interest. Preferably an at least partial protective and/or curative immune response is elicited. A protective immune response means that an animal which has been immunized will suffer less—if at all—from a disease related to the presence of said proteinaceous molecule of interest. For instance, if said proteinaceous molecule is present on a pathogen, said animal will suffer less, preferably not suffer at all, from an infection by said pathogen after the animal has been immunized. As another example, if said proteinaceous molecule is present on a tumor, or involved with tumor growth, said immunized animal will be better capable of preventing and/or counteracting (growth of) said tumor. As a result the animal will suffer less, or not at all, from a tumor-related disease.

A curative immune response means that an animal which is already suffering from a disease related to the presence of a proteinaceous molecule of interest will be better capable of counteracting (symptoms of) said disease.

The conformation of a compound is restricted in various ways. Preferably, the conformation is restricted by the formation of a linkage at at least two different sites of said compound. Each of said at least two different sites of said compound is either bound to another compound, such as a scaffold, or to another site within said compound, thus forming an internal bond. A linkage is preferably formed at a site outside an epitope. This means that amino acid residues which are part of an epitope of interest that is to be recognized by antibodies and/or T cell receptors generated by the animal's immune system are preferably not used for forming a linkage, because this will often diminish recognition of said epitope by an animal's immune system. Typically, such epitope of interest comprises an epitope capable of eliciting an immune response against a proteinaceous molecule of interest. If amino acids of such epitope of interest are used for formation of a linkage, the resulting compound will often be less—if at all—capable of eliciting an immune response which is capable of subsequently recognizing a proteinaceous molecule of interest.

When a linkage is inside an epitope it should preferably be formed between amino acid positions that are not crucial or heavily involved in antibody binding. Hence, most preferably, crucial amino acids of an epitope are not used for forming a linkage. Crucial amino acids of an epitope are amino acids whose presence is required for eliciting an immune response which is capable of recognizing a proteinaceous molecule of interest. A skilled person is well capable of determining experimentally which amino acid residues are suitable for formation of a linkage while preserving epitope recognition, and which amino acid residues should not be used.

In a preferred embodiment the conformation of a compound is restricted by the formation of at least one internal bond within said compound. In one embodiment an immunogenic compound according to the invention comprising two internal bonds is provided. In another preferred embodiment a compound according to the invention is attached to a scaffold and/or carrier. An immunogenic compound which is bound to a scaffold and/or carrier and which comprises an internal bond is most preferably provided. According to the present invention, such immunogenic compound is particularly suitable for inducing and/or enhancing immunogenic reproducibility and/or immunogenicity of a compound. It has been found that such compound is often even better capable of inducing and/or enhancing immunogenic reproducibility and/or immunogenicity, as compared to a compound having other types of linkages, such as for instance two internal bonds. An immunogenic compound which is bound to a scaffold and/or carrier and which comprises an internal bond is therefore particularly preferred. Said compound is preferably attached to a scaffold and/or carrier via at least two linkages because this particularly limits the conformation of said compound. Therefore, an immunogenic compound is preferably provided which is bound to a scaffold and/or carrier via at least two linkages and which additionally comprises at least one internal bond. The invention provides the insight that even though the conformation of current immunogenic compounds bound to a carrier or scaffold is already more restricted as compared to free compounds, a significant improvement in immunogenicity and immunogenic reproducibility is still obtained if the conformation of said scaffold-bound compound is further restricted, preferably by the formation of a second linkage. Said second linkage is preferably an internal bond, because the formation of an internal bond within a scaffold-bound compound particularly enhances immunogenicity and immunogenic reproducibility of said compound. Since a compound's conformation is already restricted when the compound is bound to a carrier or scaffold, an additional linkage—be it an internal bond or a linkage to another compound—would not be expected to have a significant effect. According to the invention, however, the formation of a second linkage does provide a significant improvement.

A method according to the invention is particularly applicable for optimizing the three-dimensional structure of an immunogenic compound which is capable of inducing an immune response against a proteinaceous molecule of interest. With a method according to the, invention the conformation of such immunogenic compound is restricted in order to force the compound to spend more time in a conformational state which closely resembles the three-dimensional structure of the corresponding epitope in said proteinaceous molecule. Hence, the native three-dimensional structure of an epitope is more closely mimicked. A method according to the invention is therefore particularly suitable for optimizing immunogenic compounds derived from epitopes with a specific three-dimensional structure in a proteinaceous molecule. Examples of such epitopes are non-linear epitopes and/or epitopes that are present in a specific three-dimensional structure found in proteins, such as for instance a loop structure. A preferred example of such loop structure is a beta-hairpin which often occurs between two antiparallel beta-strands. Beta-hairpins are often relatively easily accessible. As a result, immune responses are often directed against epitopes present in beta-hairpin sequences. Loop structures are also present in members of the cystine-knot superfamily. These members have an unusual arrangement of six cysteines linked to form a “cystine-knot” conformation, shown in FIG. 4A. The cystine-knot structure comprises 2 distorted beta-hairpin loops “above” the knot and a single beta-hairpin loop “below” the knot. Immunodominant epitopes are often found within these loops. The three-dimensional structure of epitopes present in such loop structures are preferably mimicked with a method according to the invention. The conformation of a peptide sequence derived from a hairpin-loop is preferably restricted via at least two linkages such that the conformation of said peptide sequence closely resemble the native three-dimensional structure of the hairpin-loop. Further provided is therefore a method according to the invention for inducing and/or enhancing immunogenic reproducibility and/or immunogenicity (preferably cross reactivity) of a compound, wherein said immunogenic compound comprises an amino acid sequence which is at least 50%, preferably 60%, more preferably 70, 75, 80, 85, 90 and/or 95%, homologous to a part of an amino acid sequence of a proteinaceous molecule of interest, said part having a length of at, least 8 amino acid residues, wherein said part comprises a non-linear epitope of said proteinaceous molecule and/or wherein said part comprises a sequence of at least 6 amino acid residues, preferably at least 8 amino acid residues, which is present in a loop, preferably a hairpin loop, of said proteinaceous molecule.

The three-dimensional structure of a native epitope is mimicked by restricting the conformation of an immunogenic compound, comprising a sequence at least partly derived from said epitope, with a method according to the invention. Preferably the locations of at least two linkages are chosen such that the resulting conformation of the immunogenic compound resembles the native conformation of said epitope in said proteinaceous molecule of interest. For instance, the position of an internal bond is chosen such that the conformation of the resulting compound closely resembles the native conformation of an epitope of said proteinaceous molecule of interest. Additionally, or alternatively, the position of a linkage between said immunogenic compound and a scaffold is chosen such that the conformation of the resulting compound closely resembles the native conformation of an epitope of said proteinaceous molecule of interest. The conformation of an immunogenic compound according to the invention is also influenced by the type of scaffold that is used, since the size and the shape of the scaffold influence the overall structure of the immunogenic compound. A skilled person is well capable of designing an immunogenic compound according to the invention with a conformation closely resembling the native conformation of an epitope of a proteinaceous molecule of interest. Preferably the location of at least two linkages are chosen such that a conformation is obtained which closely resembles the native conformation of an epitope of said proteinaceous molecule of interest. This is for instance schematically exemplified in FIGS. 4B and 4C. Of course, a linkage is preferably not located within an epitope of interest, because such linkage would disturb the conformation and/or accessibility of the epitope. If a scaffold is used, the kind of scaffold and the location where the scaffold is linked to an amino acid sequence of the immunogenic compound are chosen such that a conformation is obtained which closely resembles the native conformation of an epitope of said proteinaceous molecule of interest. It is for instance possible to produce several compounds with linkages at different locations and to experimentally determine the immunogenicity and/or immunogenic reproducibility of the resulting compounds. A compound with optimal immunogenic properties is preferably selected. This is for instance exemplified in example 1. In example 1 is shown how an optimal conformation is determined by varying the location of an internal bond. Of course, the method outlined in this example is not limiting the invention. It is also possible to produce several compounds with different kinds of scaffolds, either linked at identical or different locations of an amino acid sequence, and to experimentally determine the immunogenicity and/or immunogenic reproducibility of the resulting compounds.

As used herein, the term “immunogenic compound” encompasses any kind of compound capable of eliciting an immune response in a host. Preferably, but not necessarily, an immunogenic compound according to the invention comprises an amino acid sequence. The invention is now further described for the preferred embodiments wherein immunogenic compounds comprising an amino acid sequence are used.

The conformation of an immunogenic compound comprising an amino acid sequence is preferably restricted by attaching the amino acid sequence to a carrier or scaffold, either directly or indirectly, for instance via a linker, and by the formation of at least one internal bond within said amino acid sequence. Said internal bond preferably comprises a disulphide bond (also called an SS-bridge) because disulphide bonds are selectively formed between free cysteine residues without the need to protect other amino acid side chains. Furthermore, disulphide bonds are easily formed by incubating in a basic environment. Preferably a disulphide bond is formed between two cysteine residues, since their sulfhydryl groups are readily available for binding. The location of an SS-bridge within an amino acid sequence is easily regulated by regulating the location of free cysteine residues. In a particularly preferred embodiment said cysteines are located around the first and last amino acid position of the amino acid sequence, in order to optimally restrict the conformation of the amino acid sequence.

Of course, other kinds of internal bonds are also suitable for restricting the conformation of an immunogenic compound of the invention. For instance, Se—Se diselenium bonds are used. An advantage of diselenium bonds is the fact that these bonds are reduction insensitive. Hence, immunogenic compounds comprising a diselenium bond are better capable of maintaining their conformation under reducing circumstances, for instance present within an animal body. Furthermore, a diselenium bond is preferred when a free SH-group is present within the immunogenic compound, which SH-group is for instance used for a subsequent coupling reaction to a carrier. Such free SH-group is not capable of reacting with a diselenium bond. Alternatively, or additionally, a metathese reaction is used in order to form an internal bond. In a metathese reaction two terminal CC-double bonds or triple bonds are connected by means of a Ru-catalysed rearrangement reaction. Such terminal CC-double or CC-triple bonds are for instance introduced into a peptide either via alkylation of the peptide NH-groups, for instance with allyl bromide or propargyl bromide, or via incorporating a non-natural amino acid with an alkenyl- or alkynyl-containing side chain into the peptide. A metathese reaction does not occur spontaneously, but is performed with a Grubbs-catalyst.

In one embodiment an internal bond is formed using Br—SH cyclisation. For instance, an SH moiety of a free cysteine is coupled to a BrAc-moiety which is preferably present at the N-terminus of the peptide or at a lysine (RNH₂) side chain.

In a further embodiment a CO₂H-side chain of an aspartate or glutamate residue is coupled to the NH₂-side chain of a lysine residue. This way an amide bond is formed. It is also possible to form an internal bond by coupling the free CO₂H-end of a peptide to the free NH₂-end of the peptide, thereby forming an amide-bond. Alternative methods for forming an internal bond within an amino acid sequence are available, which methods are known in the art.

In principle, an internal bond is formed anywhere within an immunogenic amino acid sequence, as long as the primary, secondary and tertiary sequence of at least one epitope of interest is essentially maintained. In one preferred embodiment a linkage is formed between any one of the ten N-terminal and ten C-terminal amino acid residues of the amino acid sequence. Preferably, a linkage is formed between any one of the six N-terminal and six C-terminal amino acid residues, preferably between any one of the four N-terminal and four C-terminal amino acid residues, of the amino acid sequence. Of course, the sites that are suitable for the formation of an internal bond are dependent on the location of the epitope(s) of interest. In one preferred embodiment a linkage is formed between the first and the last amino acid residue of an immunogenic amino acid sequence.

An immunogenic compound according to the invention preferably comprises an amino acid sequence attached to a scaffold and/or a carrier because a scaffold and/or carrier enhances immunogenicity. In one preferred embodiment said amino acid sequence comprises an internal bond. An immunogenic compound comprising an amino acid sequence bound to a scaffold and/or carrier, wherein said amino acid sequence comprises at least one internal bond is therefore also herewith provided. An immunogenic compound is preferably attached to a scaffold and/or carrier via at least two linkages in order to further limit flexibility of the amino acid sequence. Most preferably, an amino acid sequence comprising at least one internal bond is attached to a scaffold or carrier via at least two linkages. This way, immunogenicity and/or immunogenic reproducibility is particularly enhanced. In a particularly preferred embodiment said scaffold comprises a (hetero)aromatic molecule with at least a first and a second reactive group as disclosed in WO 2004/077062, preferably a (hetero)aromatic molecule comprising at least two benzylic halogen substitutents. The two benzylic halogen substitutents are preferably used as first and second reactive group for coupling an amino acid sequence. An amino acid sequence is preferably coupled to a scaffold using a method according to WO 2004/077062. Briefly, a scaffold with at least a first and a second reactive group is provided. An amino acid sequence capable of reacting with said at least first and second reactive group is contacted with said scaffold under conditions allowing said amino acid sequence to react with said at least first and second reactive group to form at least two linkages between said scaffold and said amino acid sequence, wherein the formation of a first linkage accelerates the formation of a consecutive linkage. This way, conformationally constraint loop constructs are formed. An advantage of the methods and scaffolds according to WO 2004/077062 is the fact that amino acid,sequences are coupled to these scaffolds in a fast, simple and straightforward way. With a method as disclosed in WO 2004/077062 it has become possible to use unprotected peptides. Hence, laborious protection and deprotection steps are not necessary. Furthermore, the scaffolds need not be selectively functionalized. Moreover, the coupling reaction using a scaffold as disclosed in WO 2004/077062 is suitable for being performed in solution. An amino acid sequence is preferably coupled to a scaffold using a method according to WO 2004/077062 in an aqueous solution, thereby limiting or even avoiding the use of (toxic) organic solvents. Water is environment-friendly and easily removed by freeze-drying. Furthermore, many unprotected peptides are well soluble in water, as well as most salts, allowing the use of ammonium bicarbonate, one of the few volatile salts, which defines the pH to a slightly basic value of pH 7.8 to 8.0 in aqueous solution.

Since the formation of a first linkage accelerates the formation of a second linkage, the attachment of an amino acid sequence to a scaffold according to WO 2004/077062 takes place in a rapid, concerted process comprising a cascade of reactions. The formation of a first linkage, also referred to as (chemical) bond or connection, via a first reactive group increases the reactivity of a second reactive group, and so on, such that the activating effect is being ‘handed over’ from one reactive group to the next one. Said chemical reactions involve changes at functional groups while the molecular skeleton of the scaffold remains essentially unchanged. For example, a scaffold molecule as used in WO 2004/077062 comprising at least two reactive groups is capable of reacting with an amino acid sequence such that the reactive groups of the scaffold become involved in the new linkages with the amino acid sequence while the core structure or skeleton of the scaffold does not participate directly in the coupling.

In one embodiment, a synthetic scaffold comprising at least two identical reactive groups is coupled to one or more immunogenic peptides. Said one or more immunogenic peptides are further provided with a second linkage, preferably an internal bond, either before or after coupling of the peptide(s) to the scaffold. Suitable peptides comprise all possible peptides capable of reacting with at least two reactive groups on a scaffold to form at least two linkages or bonds between said peptide(s) and said scaffold, which typically results in a looped or cyclic peptide on a scaffold. Speaking in terms of organic chemistry, the essence of such a bond formation is charge attraction and electron movement. In a preferred embodiment, the coupling reaction between an amino acid sequence and a scaffold involves a nucleophilic substitution reaction wherein an amino acid sequence with a free nucleophilic functionality reacts with a scaffold. A nucleophile typically shares an electron pair with an electrophile in the process of bond formation. In other words, a nucleophile is seeking a center of electron deficiency (an atom) with which to react. Nucleophiles, (‘nucleus-loving’) can be negatively charged or uncharged, and include for example heteroatoms other than carbon bearing a lone pair, or pi electrons in any alkene or alkyne. Electrophiles (“electron-loving’) are electrically neutral or positively charged and have some place for the electrons to go, be it an empty orbital (as in BH3) or a potentially empty orbital. In a preferred embodiment said nucleophilic functionality comprises a thiol or sulfhydryl group. Thiols are effective nucleophiles for substitution at saturated carbon atoms. It is in general not difficult to provide an amino acid sequence with a nucleophilic functionality. For example, an amino acid sequence is easily functionalised with a thiol moiety by incorporating a cysteine residue in the amino acid sequence.

A common characteristic of a nucleophilic reaction that takes place on saturated carbon, is that the carbon atom is almost always bonded to a heteroatom, defined herein as an atom other than carbon or hydrogen. Furthermore, the heteroatom is usually more electronegative than carbon and is also the so-called leaving group (L) in the substitution reaction. The leaving group departs with the electron pair by which it was originally bound to the carbon atom. In a preferred embodiment, a scaffold is used which contains at least two leaving groups in order to facilitate the formation of at least two bonds with at least one amino acid sequence. The ease with which a leaving group departs is related to the basicity of that group; weak bases are in general good leaving groups because they are able to accommodate the electron pair effectively. The reactivity of a reactive group is largely determined by the tendency of a leaving group to depart. Another factor which has some bearing on reactivity of a reactive group is the strength of the bond between the leaving group and the carbon atom, since this bond must break if substitution is to occur.

Thus, in a preferred embodiment, a scaffold comprising at least two reactive groups each comprising a good leaving group is used in a method according to the invention. Good leaving groups are in general the conjugate bases of strong acids. Important leaving groups are the conjugate bases of acids with pKa values below 5. Particularly interesting leaving groups include halide ions such as I—, Br—, and Cl—. A carbon-halogen (C—X) bond in an alkyl halide is polarised, with a partial positive charge on the carbon and a partial negative charge on the halogen. Thus, the carbon atom is susceptible to attack by a nucleophile (a reagent that brings a pair of electrons) and the halogen leaves as the halide ion (X—), taking on the two electrons from the C—X bond. Therefore, in one embodiment an amino acid sequence is coupled to a reactive group of a scaffold, the reactive group comprising a carbon atom susceptible to attack by a nucleophile wherein said reactive group comprises a carbon-halogen bond. In a preferred embodiment, a scaffold comprising at least two of such reactive groups is used to react with a di-SH functionalised peptide as nucleophile. Provided is a method for obtaining an immunogenic compound comprising a scaffold with at least one looped peptide structure, said method comprising contacting said scaffold with at least one peptide, wherein said scaffold comprises a halogenoalkane, where after the coupled peptide is provided with a second linkage, preferably an internal bond. Alternatively, the peptide is provided with the second linkage, preferably an internal bond, before it is coupled to the halogenoalkane. Halogenoalkanes (also known as haloalkanes or alkyl halides) are compounds containing a halogen atom (fluorine, chlorine, bromine or iodine) joined to one or more carbon atoms in a chain. Particularly suitable are dihaloscaffolds, comprising two halogen atoms, and tri- and tetrahaloscaffolds for the synthesis of conformationally constraint compounds, like for example peptide constructs consisting of one or more looped peptide segments bound to said scaffold, wherein the looped peptide segments are further provided with a second linkage, preferably with an internal bond.

In general, a good leaving group is electronegative to polarize the carbon atom, it is stable with an extra pair of electrons once it has left, and is polarizable, to stabilize the transition state. With the exception of iodine, all of the halogens are more electronegative than carbon. Chlorine and bromine have fairly similar electronegativities and polarize the bond with the carbon fairly equally. When ionized, both are very weak bases with Br— being the weaker one of the two. Bromide ion is also more polarizable due to its larger size. Therefore, an immunogenic peptide is preferably bound to a scaffold comprising at least two Cl atoms, more preferably bound to a scaffold comprising at least one Cl atom and at least one Br atom and even more preferably bound to a scaffold comprising at least two Br atoms.

In a preferred embodiment, a conformationally restricted amino acid sequence is bound to a scaffold which comprises an allylic system. In an allylic system, there are at least three carbon atoms, two of which are connected through a carbon-carbon double bond. In a preferred embodiment, the formation of a bond or linkage between a scaffold and an immunogenic peptide occurs via an allylic substitution reaction. An allylic substitution reaction refers to a substitution reaction occurring at position 1 of an allylic system, the double bond being between positions 2 and 3. The incoming group becomes attached to the same atom 1 as the leaving group, or the incoming group becomes attached at the relative position 3, with movement of the double bond from ⅔ to ½. The reaction rate of allylic substitutions is every high, because the allyl cation reaction intermediate, a carbon atom bearing a positive charge attached to a doubly-bonded carbon, is unusually stable. This is because an allylic cation is a resonance hybrid of two exactly equivalent structures. In either of the contributing structures, there is an empty p orbital with the pi cloud of the electron-deficient carbon. Overlap of this empty p orbital with the pi cloud of the double bond results in delocalisation of the pi electrons, hereby providing electrons to the electron-deficient carbon and stabilizing the cation.

Even more preferred is a scaffold comprising at least two allylic halogen atoms. Due to electron delocalisation, allyl halides tend to undergo ionization very readily to produce a carbocation and a halide ion, such that breaking the carbon halide bond is rapid. In a further embodiment of the invention, a carbon-oxygen double bond (i.e. a carbonyl group) is present in a scaffold. Similarly to the allylic system, resonance structures are formed which contribute to stabilization of a carbocation. For example, a scaffold comprises two or more reactive groups comprising the structure-C(O)—CH2-halogen.

Furthermore, in a nucleophilic substitution reaction, the structure of the substrate plays just as important role as the nature of the leaving group. For example, if a nucleophile attacks the backside of the carbon, the reaction proceeds unhindered if the leaving group is bonded to a methyl, where the hydrogens leave enough surface to attack the carbon. As that carbon becomes more substituted, larger groups hinder the path the nucleophile must take to displace the leaving group. For these reasons, it is also advantageous that a scaffold comprise at least two halomethyl groups.

In one embodiment, a scaffold comprises a conjugated polyene, also known as aromatic compound, or arene, which is provided with at least two reactive groups. An aromatic compound is flat, with cyclic clouds of delocalised pi electrons above and below the plane of the molecule. Preferably, a molecular scaffold is used which comprises at least two benzylic halogen substituents, like for instance halomethyl groups. Suitable examples include, but are not limited, to di(halomethyl)benzene, tri(halomethyl)benzene or tetra(halomethyl)benzene and derivatives thereof. An advantage of a benzylic halogen substituent is mainly to be sought in the special stability associated with the resonance of conjugated polyenes known as aromatic compounds; a benzylic halogen atom has an even stronger tendency to leave a carbon on which a nucleophilic substitution reaction takes place.

A preferred embodiment of the invention therefore provides a method wherein an amino acid sequence is bound via at least two linkages to a scaffold comprising a (hetero)aromatic molecule with at least two benzylic halogen substituents and wherein the conformation of said amino acid sequence is further restricted, preferably via an internal bond. Said internal bond preferably comprises an internal disulfide bond. Said amino acid sequence is preferably bound to the (hetero)aromatic molecule using two free cysteine thiols of the amino acid sequence. Preferably, said scaffold is a halomethylarene, preferably selected from the group consisting of bis(bromomethyl)benzene, tris(bromomethyl)benzene and tetra(bromomethyl)benzene, or a derivative thereof. More preferably said scaffold is selected from the group consisting of ortho-, meta- and para-dihaloxyleen and 1,2,4,5 tetra halodurene. Said scaffold most preferably comprises meta-1,3-bis(bromomethyl)benzene (m-T2), ortho-1,2-bis(bromomethyl)benzene (o-T2), para-1,4-bis(bromomethyl)benzene (p-T2), meta-1,3-bis(bromomethyl)pyridine (m-P2), 2,4,6-tris(bromomethyl)mesitylene (T3), meta-1,3-bis(bromomethyl)-5-azidobenzene (m-T3-N3) and/or 1,2,4,5 tetrabromodurene (T4).

The invention thus provides a method for inducing and/or enhancing the immunogenicity and/or immunogenic reproducibility of an amino acid sequence comprising coupling said amino acid sequence, which amino acid sequence preferably comprises two free cysteine thiols, to a (hetero)aromatic molecule with at least two benzylic halogen substituents, preferably a halomethylarene, more preferably bis(bromomethyl)benzene, tris(bromomethyl)benzene and/or tetra(bromomethyl)benzene or a derivative thereof, and forming at least one internal bond within said amino acid sequence. Said internal bond is either formed before, or after coupling of the amino acid sequence to the scaffold. Preferably, said internal bond is formed after the coupling reaction has been performed. Said amino acid sequence is preferably coupled to the (hetero)aromatic molecule via at least two linkages in order to particularly limit the conformation of said amino acid sequence. Most preferably, an amino acid sequence is provided with an internal bond and coupled to m-T2, o-T2, p-T2, m-P2, T3, m-T3-N3 and/or T4.

Suitable molecular scaffolds also include polycyclic aromatic compounds with smaller or larger ring structures. However, suitable scaffolds are not limited to hydrocarbons. In contrast, a heterocyclic aromatic scaffold—a cyclic molecule with at least one atom other than carbon in the ring structure, most commonly nitrogen, oxygen or sulfur—is also suitable. Examples include pyrrole, furan, thiophene, imidazole, oxazole, thiazole, pyrazole, -3-pyrroline, pyridine, pyrimidine and derivatives thereof. Preferred heterocyclic aromatic scaffolds include but are not limited to those comprising at least two halomethyl groups. A preferred scaffold is meta-dibromo-pyridine.

In another embodiment, an amino acid sequence is coupled to a scaffold that is based on or which consists of multiple ring aromatic structures, such as fused-ring aromatic compounds. Two aromatic rings that share a carbon-carbon bond are said to be fused. Suitable fused-ring aromatic scaffolds include for example naphthalene, anthracene or phenanthrene and derivatives thereof, provided that they contain at least two reactive groups. In a preferred embodiment, a fused-ring aromatic scaffold comprises at least two reactive groups wherein each group contains a highly reactive benzylic halogen atom, for example a halomethyl group.

Molecules comprising multiple aromatic or conjugated systems wherein the systems do not share a pair of carbon atoms are also useful as scaffold molecule. For example, a scaffold comprises a multi-ring or fused ring structure, for instance a scaffold wherein aromatic, e.g. benzene, rings are connected directly via a carbon-carbon bond is used. Alternatively, said rings are connected via a linker comprising at least one atom. Examples of suitable scaffolds are given in FIGS. 1-3. From a commercial point of view, a scaffold according to the invention is preferably commercially available at a relatively low cost and obtainable in large quantities. For example, the dibromo scaffold 1,3-bis(bromomethyl)benzene is currently being sold for only around 5 euro per gram.

Typically, a peptide molecule for use in a method according to the invention is a synthetic peptide, for instance obtained using standard peptide synthesis procedures. Synthetic peptides are obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is for instance attached to a cross-linked polystyrene resin via an acid labile bond with a linker molecule. This resin is insoluble in the solvents used for synthesis, making it relatively simple and fast to wash away excess reagents and by-products. Suitable peptides comprise peptides of various length. As is exemplified herein, oligopeptides ranging from as small as 3 amino acids in length to polypeptides of 27 residues have been successfully used in a method provided. The maximal length or size of a suitable peptide or essentially depends on the length or size which can be achieved using peptide synthesis. In general, peptides of up to 30 amino acid residues can be synthesized without major problems.

Coupling of an amino acid sequence to a scaffold according to WO 2004/077062 allows for the use of unprotected amino acid sequences. The only functionality that cannot be present in unprotected form is the cysteine SH, as it will be involved in the coupling reaction. In one embodiment of the invention an amino acid sequence is used which, besides two free cysteine residues for coupling to a scaffold, comprises at least two more additional cysteine (Cys)residues. To prevent unwanted participation of these additional Cys thiol groups in the coupling reaction, a simple approach is for instance to use Fmoc-Cys(Acm) (Fmoc-acetamidomethyl-L-cysteine) for introduction of a protected Cys residue during the course of peptide synthesis. Alternatively, Fmoc-Cys(StBu)-OH is used, and/or the corresponding Boc amino acids. The Acm or StBu group is not removed during the course of the normal TFA deprotection-cleavage reaction but requires oxidative (I2/VitC) treatment in case of Acm group, or reductive treatment (BME (excess) or 1,4-DTT (excess)) in case of StBu group to give the reduced sulfhydryl form of the peptide, which can either be used directly or subsequently oxidized to the corresponding cystinyl peptide. In one embodiment, a peptide is used which contains at least one Cys derivative, such as Cys(Acm) or Cys(StBu), to allow selective unmasking of a Cys-thiol group. Selective unmasking of a Cys-thiol group allows to make the Cys-thiol group available for reacting at a desired moment, such as following completion of the coupling reaction between a scaffold and a peptide. This is for instance very attractive for forming an internal bond within the peptide after the peptide has been bound to a scaffold. For example, in a preferred embodiment a linear peptide is synthesized, comprising two unprotected Cys residues and two protected Cys derivatives at other positions. Thereafter, the di-SH functionalized peptide is coupled to a (hetero)aromatic scaffold comprising at least two benzylic halogen substituents, resulting in the structural fixation of a looped peptide on the scaffold. Subsequently, the two Cys-derivatives are unmasked and used for forming an internal disulfide bridge.

Further provided herein is a compound comprising an amino acid sequence bound to a scaffold and/or a carrier via at least two linkages, wherein said amino acid sequence comprises at least one internal bond. In one embodiment said internal bond comprises an SS-bridge, preferably between two cysteine residues of said amino acid sequence because their sulfhydryl groups are readily available for binding. Said cysteines are preferably located around the first and last amino acid position of the amino acid sequence in order to at least partially avoid the formation of free rotating peptide ends. Said compound preferably comprises an immunogenic compound and/or a protein mimic. A non-limiting example of a protein mimic according to the invention is a peptide that mimics the binding properties of a protein (for instance receptor activating or receptor inhibiting). As explained before, the sites where a linkage is formed are dependent on the position of at least one epitope of interest within said amino acid sequence. In general, a linkage is not formed at a site within an epitope sequence, because that would diminish immunogenicity.

An immunogenic compound according to the invention preferably comprises an amino acid sequence that is bound to a scaffold via at least two linkages, because this particularly limits the conformation of said compound. As explained before, said scaffold preferably comprises a (hetero)aromatic molecule comprising at least two benzylic halogen substitutents, preferably a halomethylarene. Preferred scaffolds are bis(bromomethyl)benzenes, tris(bromomethyl)benzenes and tetra(bromomethyl)benzenes, or derivatives thereof. An immunogenic compound according to the invention preferably comprises a scaffold selected from the group consisting of ortho-, meta- and para-dihaloxyleen and 1,2,4,5 tetra halodurene, preferably selected from the group consisting of meta-1,3-bis(bromomethyl)benzene (m-T2), ortho-1,2-bis(bromomethyl)benzene (o-T2), para-1,4-bis(bromomethyl)benzene (p-T2), meta-1,3-bis(bromomethyl)pyridine (m-P2), 2,4,6-tris(bromomethyl)mesitylene (T3), meta-1,3-bis(bromomethyl)-5-azidobenzene (m-T3-N3) and 1,2,4,5 tetrabromodurene (T4).

An immunogenic compound according to the invention is particularly suitable for inducing and/or enhancing a desired immune response. In one embodiment an immunogenic compound according to the invention is combined with a pharmaceutically acceptable carrier, adjuvant, diluent and/or excipient in order to enhance antibody production or a humoral response. Examples of suitable carriers for instance comprise keyhole limpet haemocyanin (KLH), serum albumin (e.g. BSA or RSA) and ovalbumin. Many suitable adjuvants, oil-based and water-based, are known to a person skilled in the art. In one embodiment said adjuvant comprises Specol. In another embodiment, said suitable carrier comprises a solution like for example saline.

An immunogenic composition comprising an immunogenic compound according to the invention and a pharmaceutically acceptable carrier, adjuvant, diluent and/or excipient is therefore also provided. Said immunogenic composition preferably comprises a vaccine, capable of inducing a protective immune response. Alternatively, or additionally, an immunogenic compound according to the invention is used for inducing and/or enhancing a curative immune response in order to treat a patient suffering from a disease. An immunogenic compound according to the invention for use as a medicament and/or vaccine is also herewith provided. Dose ranges of compounds according to the invention to be used in the prophylactic and/or therapeutic applications as described herein are designed on the basis of rising dose studies in the clinic in clinical trials for which rigorous protocol requirements exist. Typically, doses vary between 0.01-1000 μg/kg body weight, particularly about 0.1-100 μg/kg body weight.

An immunogenic compound according to the invention preferably comprises an amino acid sequence derived from a proteinaceous molecule of interest, in order to elicit an immune response against said proteinaceous molecule of interest. In one embodiment said proteinaceous molecule of interest is selected from the group consisting of the cystine-knot family, transmembrane proteins, TNF-alpha, HGF/SF, FGF-beta, interleukins, IL-5, chemokines, G-protein-coupled receptors, CCR5, CXCR4, IGF, LMF, endothelin-1, VIP, CGRP, PIF, EGF, TGF-alpha, the ErbB family, HER1/EGF-R, HER2/neu, HER3, HER4, p53, corticotrophin RF, ACTH, parathyroid hormone, CCK, substance P, NPY, GRP, neurotrophine, angiotensin-2, angiogenin, angiopoietin, neurotensine, SLCLC, SARS-derived proteins, HIV-derived proteins, papillomavirus-derived proteins and FMDV. An immune response against any of these proteinaceous molecules of interest is preferably elicited and/or enhanced in order to prevent and/or counteract a disorder related to the presence of said proteinaceous molecule of interest. Further provided is therefore a use of an immunogenic compound according to the invention for the preparation of a medicament and/or vaccine against a disorder related to the presence of a member of the cysteine-knot family, a transmembrane protein, TNF-alpha, HGF/SF, FGF-beta, an interleukin, IL-5, a chemokine, a G-protein-coupled receptor, CCR5, CXCR4, IGF, LMF, endothelin-1, VIP, CGRP, PIF, EGF, TGF-alpha, the ErbB family, HER1/EGF-R, HER2/neu, HER3, HER4, p53, corticotrophin RF, ACTH, parathyroid hormone, CCK, substance P, NPY, GRP, neurotrophine, angiotensin-2, angiogenin, angiopoietin, neurotensine, SLCLC, SARS-derived protein, HIV-derived protein, papillomavirus-derived protein and/or FMDV.

In a particularly preferred embodiment an amino acid sequence of an immunogenic composition according to the invention is derived from a member of the cystine-knot family. Members of the cystine-knot (Cys-knot) family have an unusual arrangement of six cysteines linked to form a “cystine-knot” conformation. The active forms of these proteins are dimers, either homo- or heterodimers. Because of their shape, there appears to be an intrinsic requirement for the cystine-knot growth factors to form dimers. This extra level of organization increases the variety of structures built around this simple structural motif. Many members of the Cys-knot family are growth factors.

In the crystal structures of transforming growth factor-beta 2 (TGF-beta2), platelet-derived growth factor (PDGF), nerve growth factor (NGF) and human chorionic gonadotropin (hCG), 6 conserved cysteine residues (CysI to CysVI in sequence order) form 3 disulphide bonds arranged in a knot-like topology. The two disulphide bonds between CysII and CysV ([CysII-V]) and between CysIII and CysVI ([CysIII-VI]) form a ring-like structure of 8 amino acids through which the remaining disulphide bond (between CysI and CysIV) penetrates (see FIG. 4A). The sulfur (S) atoms of the conserved cysteines I to VI that are involved in the disulphide bonds are typically referred to as S1 to S6. Cystine knot domains with more than 6 cysteine residues can be found. The “extra” cysteine residues are normally used to create further disulphide bonds within the cystine knot domain or interchain disulphide bonds, during dimerisation. However, based on homology and topology it is always possible to indicate which cysteines represent the six conserved residues CysI to CysVI (see further below).

A similar knotted arrangement of disulphide bonds has been noted in the structures of some enzyme inhibitors and neurotoxins that bind to voltage-gated Ca²⁺ channels McDonald et al. 1993, Cell 73 421-424). In those sequences, however, the cystine topology differs: Cys[III-VI] penetrates a macrocyclic ring formed by Cys[I-IV] and Cys[II-V]. Thus, cystine-knot proteins fall into 2 structural classes growth factor type and inhibitor-like cystine knots.

The cystine-knot growth factor superfamily is divided into subfamilies, which include the glycoprotein hormones (e.g. follicle stimulating hormone (FSH)), the transforming growth factor beta (TGF-beta) proteins (e.g. bone morphogenetic protein 4), the platelet-derived growth factor-like (PDGF-like) proteins (e.g. platelet derived growth factor A), nerve growth factors (NGF) (e.g. brain-derived neurotrophic factor) (see also Tables 1 and 2).

All growth factor cystine knot structures have a similar topology, with 2 distorted beta-hairpin (beta-1 and beta-3) loops “above” the knot and a single (beta-2) loop “below” the knot. The beta-1 loop is formed by the stretch of amino acids between CysI and CysII; the beta-2 loop is formed by the amino acids between CysIII and CysIV and the beta-3 loop is formed by the amino acids between CysIV and CysV (see FIG. 4A). The sizes of the hairpin loops (i.e. the number of amino, acids between the indicated cysteines) can vary significantly between family members.

In a particularly preferred embodiment an immunogenic compound according to the present invention comprises an amino acid sequence that is capable of inducing and/or enhancing an immune response in an animal against a member of the glycoprotein hormone-beta (GLHB) subfamily, the platelet-derived growth factor (PDGF) subfamily, the transforming growth factor (TGF) subfamily, the nerve growth factor (NGF) subfamily or the glycoprotein hormone-alpha (GLHA) subfamily. Said amino acid sequence preferably comprises a sequence which is at least 50%, more preferably at least 60%, more preferably at least 70%, more preferably at least 80%, more preferably at least 85%, more preferably at least 90%, more preferably at least 95%, most preferably at least 98% homologous to at least part of said cystine-knot protein family member, said part having a length of at least 8 amino acid residues. The higher the homology, the more specifically an elicited immune response will be directed against said cystine-knot protein family member. These subfamilies play an important role in the formation and proliferation of many different types of cancers. Therefore, eliciting an immune response specifically directed against at least one member of these subfamilies is useful for preventing and/or counteracting this kind of diseases. Moreover, some members of these subfamilies are involved in fertility regulation (hCG, FSH).

In a particularly preferred embodiment the immunogenicity and/or immunogenic reproducibility of an immunogenic compound capable of inducing an immune response against VEGF is enhanced. Eliciting and/or enhancing an immune response against VEGF (in particular VEGF-A/B, VEGF-C and/or VEGF-D) counteracts (lymph)angiogenesis. This is for instance desired when an individual is suffering from, or at risk of suffering from, a tumor-related disease. Tumor growth requires (lymph)angiogenesis, which involves the formation of new blood (or lymphatic) vessels, in order to carry nutrients to the site of the tumor, to transport waste material from the tumor, and to spread. Counteracting (lymph)angiogenesis therefore hampers tumor growth and spread. (Lymph)angiogenesis involves the action of endogenous growth hormones such as vascular endothelial growth factor (VEGF-A/B, VEGF-C and/or VEGF-D). Hence, counteracting the action of such growth factor indirectly counteracts tumor growth because angiogenesis is at least in part prevented.

Likewise, placental growth factor (PlGF) is involved in angiogenesis. Eliciting an immune response against PlGF therefore counteracts angiogenesis and, indirectly, counteracts tumor growth. Since PlGF is primarily involved in angiogenesis in tumour tissue but not, or to a significantly lower extent, in angiogenesis in normal tissue, therapy involving eliciting and/or enhancing an immune response against PlGF will particularly avoid negative side-effects.

In a further preferred embodiment the immunogenicity and/or immunogenic reproducibility of an immunogenic compound capable of inducing an immune response against hCG is enhanced. hCG is often overexpressed in tumour tissue. Eliciting an immune response against hCG therefore attacks tumour tissue. Since hCG is normally only expressed during pregnancy, negative side effects are at least in part avoided in non-pregnant individuals.

Furthermore, human epidermal growth factor receptor (HER) and hepatocyte growth factor/stimulating factor (HGF/SF) are often overexpressed in tumour tissue. An immune response against these proteins therefore also attacks tumor tissue. An immune response against VEGF, hCG, PlGF, HER and/or HGF/SF is preferably elicited with an amino acid sequence that is at least partly derived from said proteins. As explained before, the amino acid sequences are preferably—but not necessarily—optimized using for instance a TDK-Alascan method and/or replacement net mapping. This way immunogenicity is enhanced. Further provided is therefore an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98%, homologous to at least part of the amino acid sequence of VEGF, hCG, PlGF, HER and/or HGF/SF, said part having a length of at least 8 amino acid residues. A use of said immunogenic compound for the preparation of a medicament and/or vaccine against a tumor-related disease is also provided, as well as a method for vaccinating an animal against a tumor-related disease, comprising administering to said animal a suitable dose of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of VEGF, hCG, PlGF, HER and/or HGF/SF, said part having a length of at least 8 amino acid residues. A use of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of hCG, said part having a length of at least 8 amino acid residues, for the preparation of a medicament and/or vaccine for fertility regulation is also provided.

Whereas PlGF primarily involved in angiogenesis in tumour tissue, VEGF (VEGF-A/B, VEGF-C and VEGF-D) plays an important role in angiogenesis in other (non-tumour) tissue as well. Also in non-tumour-related situations it is often desired to counteract angiogenesis, for instance in case of inocular retinopathy, during which undesired angiogenesis in the eye results in loss of sight. Further provided is therefore a use of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of VEGF, said part having a length of at least 8 amino acid residues, for the preparation of a medicament and/or vaccine against angiogenesis. Another embodiment provides a method for vaccinating an animal against angiogenesis, comprising administering to said animal a suitable dose of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of VEGF, said part having a length of at least 8 amino acid residues. In case of inocular retinopathy said immunogenic compound according to the invention is for instance administered to an individual in order to elicit and/or enhance an immune response against VEGF-A or VEGF-B. Additionally, or alternatively, the eye is provided with anti VEGF-A or anti VEGF-B antibodies or T cells derived from the individual or a non-human animal after immunization with said immunogenic compound according to the invention. If said immunogenic compound is administered to a non-human animal, harvested antibodies or T cells are preferably further processed in order to adapt them to human use, using methods known in the art. For instance, the six hypervariable regions from the heavy and light chains of a non-human antibody are incorporated into a human framework sequence and combined with human constant regions.

Hepatocyte growth factor receptor/stimulating factor (HGF/SF) is primarily involved with the formation of metastases once a tumour is present. Especially in case of prostate cancer HGF/SF plays an important role. In order to counteract the formation of metastases it is thus particularly preferred to counteract the action of HGF/SF. Further provided is therefore a use of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably atleast 95%, most preferably at least 98% homologous to at least part of the amino acid sequence of HGF/SF, said part having a length of at least 8 amino acid residues, for the preparation of a medicament and/or vaccine against the formation of metastases during a tumor-related disease, preferably during prostate cancer. Also provided is a method for vaccinating an animal against the formation of metastases during a tumor-related disease, preferably prostate cancer, comprising administering to said animal a suitable dose of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of HGF/SF, said part having a length of at least 8 amino acid residues.

Human epidermal growth factor receptor (HER or ErbB (or EGF-R in case of HER-1 or neu in case of HER-2)) is overexpressed by a wide variety of tumours, such as for instance breast tumor. Since HER is overexpressed by many tumors, immunization, against HER thus provides a general therapy for a broad spectrum of tumor-related diseases. Further provided is therefore a use, of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of HER, said part having a length of at least 8 amino acid residues, for the preparation of a medicament and/or vaccine against a tumor-related disease, preferably breast cancer. Also provided is a method for vaccinating an animal against a tumor-related disease, preferably breast cancer, comprising administering to said animal a suitable dose of an immunogenic compound according to the invention, wherein said immunogenic compound comprises an amino acid sequence comprising a sequence which is at least 50%, preferably at least 60%, more preferably at least 70%, more preferably at least 75%, more preferably at least 80%, more preferably at least 85%, more preferably at least 90%, more preferably at least 95%, most preferably at least 98% homologous to at least part of the amino acid sequence of HER, said part having a length of at least 8 amino acid residues.

Further provided are means and methods for generating immunogenic compounds according to the invention. One embodiment provides a method for preparing a compound according to the invention, the method comprising:
providing a scaffold comprising at least a first and a second reactive group;
providing at least one molecule capable of reacting with, said at least first and second reactive group, said molecule comprising an amino acid sequence;
contacting said scaffold with said at least one molecule to form at least two linkages between said scaffold and said at least one molecule in a coupling reaction, whereby the formation of a linkage accelerates the formation of a consecutive linkage, preferably wherein said coupling reaction is performed in solution, more preferably in an aqueous solution; and
allowing, inducing and/or enhancing the formation of an internal bond within said molecule and/or a third linkage between said molecule and another, moiety. Said molecule preferably comprises an amino acid sequence. Said internal bond preferably comprises an SS-bridge. As stated before, said SS-bridge is preferably a bond between two cysteine residues, which cysteine residues are preferably located around the N-terminal and C-terminal ends of the amino acid sequence.

Said molecule is preferably coupled to at least one of the scaffolds mentioned above. Hence, a method for preparing a compound according to the invention is provided wherein said scaffold comprises a (hetero)aromatic molecule, preferably a (hetero)aromatic molecule comprising at least two benzylic halogen substitutents, more preferably a halomethylarene, said halomethylarene preferably being selected from the group consisting of bis(bromomethyl)benzene, tris(bromomethyl)benzene and tetra(bromomethyl)benzene, or a derivative thereof. In one embodiment said scaffold is selected from the group consisting of ortho-, meta- and para-dihaloxyleen and 1,2,4,5 tetra halodurene, preferably meta-1,3-bis(bromomethyl)benzene (m-T2), ortho-1,2-bis(bromomethyl)benzene (o-T2), para-1,4-bis(bromomethyl)benzene (p-T2), meta-1,3-bis(bromomethyl)pyridine (m-P2), 2,4,6-tris(bromomethyl)mesitylene (T3), meta-1,3-bis(bromomethyl)-5-azidobenzene (m-T3-N3) or 1,2,4,5 tetrabromodurene (T4).

Immunogenic compounds according to the invention are particularly suitable for the production of antibodies, T cells and B cells, using a non-human animal. Further provided is therefore a method for producing antibodies, T cells and/or B cells, comprising:
immunizing a non-human animal with an immunogenic compound according to the invention and/or an immunogenic composition according to the invention, and
harvesting antibodies, T cells and/or B cells capable of specifically binding said immunogenic compound from said animal. A preferred embodiment further comprises producing monoclonal antibodies using said antibody obtained from said animal. Methods and protocols for immunizing non-human animals and harvesting antibodies, T cells and/or B cells, as well as isolating antibodies of interest and producing monoclonal antibodies, are well known in the art and need no further explanation here.

In a preferred embodiment the elicited antibodies, T cells and/or B cells are further used for human benefit. For instance, the genes encoding the Ig heavy and/or light chains are isolated from a harvested B cell and expressed in a second cell, such as for instance cells of a Chinese hamster ovary (CHO) cell line. Said second cell, also called herein a producer cell, is preferably adapted to commercial antibody production. Proliferation of said producer cell results in a producer cell line capable of producing antibodies of interest. Preferably, said producer cell line is suitable for producing compounds for use in humans. Hence, said producer cell line is preferably free of pathogenic agents such as pathogenic micro-organisms.

Alternatively, or additionally, nucleic acid encoding the T cell receptor is isolated from a harvested T cell of:interest and incorporated into naive (preferably human) T cells. The T cells are preferably cultured in order to obtain a T cell line.

The invention is further explained in the following examples. These examples do not limit the scope of the invention, but merely serve to clarify the invention.

### BRIEF DESCRIPTION OF THE DRAWINGS

FIG. 1: Aromatic scaffolds with ortho-, meta-, or para-positioning of two halomethyl groups. Hal refers to chlorine, bromo, or iodine atoms.
1,2-bis(halomethyl)benzene and other regioisomers
3,4-bis(halomethyl)pyridine (X═N) and other regioisomers
3,4-bis(halomethyl)pyridazine (X═N) and other regioisomers
4,5-bis(halomethyl)pyrimidine (X═N) and other regioisomers
4,5-bis(halomethyl)pyrazine (X═N) and other regioisomers
4,5-bis(halomethyl)-1,2,3-triazine (X═N) and other regioisomers
5,6-bis(halomethyl)-1,2,4-triazine (X═N) and other regioisomers
3,4-bis(halomethyl)pyrrole (X═N), -furan (X═O), -thiophene (X═S) and other regioisomers
4,5-bis(halomethyl)imidazole (X═N,N), -oxazole (X═N,O), -thiazol (X═S) and other regioisomers
4,5-bis(halomethyl)-3H-pyrazole (X═N,N), -isooxazole (X═N,O), -isothiazol (X═S) and other regioisomers
1,2-bis(bromomethylcarbonylamino)benzene (X₁═NH, X₂═O)
2,2′-bis(halomethyl)biphenylene
2,2″-bis(halomethyl)terphenylene
1,8-bis(halomethyl)naphthalene
1,10-bis(halomethyl)anthracene
Bis(2-halomethylphenyl)methane

FIG. 2: Aromatic scaffolds with ortho-, meta-, or para-positioning of three halomethyl groups:
1,2,3-tris(halomethyl)benzene and other regioisomers
2,3,4-tris(halomethyl)pyridine (X═N) and other regioisomers
2,3,4-tris(halomethyl)pyridazine (X═N) and other regioisomers
3,4,5-tris(halomethyl)pyrimidine (X═N) and other regioisomers
4,5,6-tris(halomethyl)-1,2,3-triazine (X═N) and other regioisomers
2,3,4-tris(halomethyl)pyrrole (X═N), -furan (X═O), -thiophene (X═S) and other regioisomers
2,4,5-bis(halomethyl)imidazole (X═N,N), -oxazole (X═N,O), -thiazol (X═S) and other regioisomers
3,4,5-bis(halomethyl)-1H-pyrazole (X═N,N), -isooxazole (X═N,O), -isothiazol (X═S) and other regioisomers
2,4,2′-tris(halomethyl)biphenylene
2,3′,2″-tris(halomethyl)terphenylene
1,3,8-tris(halomethyl)naphthalene
1,3,10-tris(halomethyl)anthracene
Bis(2-halomethylphenyl)methane

FIG. 3: Aromatic scaffolds with ortho-, meta-, or para-positioning of four bromomethyl groups.
1,2,4,5-tetra(halomethyl)benzene and other regioisomers
1,2,4,5-tetra(halomethyl)pyridine (X═N) and other regioisomers
2,4,5,6-tetra(halomethyl)pyrimidine (X₁═X₂═N) and other regioisomers
2,3,4,5-tetra(halomethyl)pyrrole (X═NH), -furan (X═O), -thiophene (X═S) and other regioisomers
2,2′,6,6′-tetra(halomethyl)biphenylene
2,2″,6,6″-tetra(halomethyl)terphenylene
2,3,5,6-tetra(halomethyl)naphthalene
2,3,7,8-tetra(halomethyl)anthracene
Bis(2,4-bis(halomethyl)phenyl)methane (X═CH₂)

FIG. 4: Schematic representation of the B3-loop of Cys-knot growth factor family members and peptidomimetics thereof. Panel A shows the general loop-structure of the various members of the Cys-knot protein family. Panel B shows the. B3-loop including residues CysIV and CysV. Panel C shows the structural design of a peptidomimetic of the invention wherein two cysteines are introduced in the polypeptide such that covalent attachment of the polypeptide via these cysteines to a scaffold (indicated as T) induces the peptide to adopt a conformation which resembles the secondary structure of the B3-loop in the native protein

FIG. 5: Antibody responses from vaccination experiments with. FSH-β3 loop mT2/SS CLIPS-peptides

FIG. 6: Antibody responses from vaccination experiments with linear, single-constrained (T2 or SS) and double-constrained (T2 and SS) peptides derived from the FSH-β3 loop

FIG. 7: ELISA competition studies with anti-peptide antisera obtained by immunization experiments with linear, single-constrained (T2 only) and double-constrained (T2 and SS) peptides derived from the VEGF-A β5-loop-β6 segment.

FIG. 8: ELISA competition studies with linear, single-constrained (T2 only) and double-constrained (T2 and SS) peptides derived from the ECL2a-loop of the CCR5 co-receptor (GPCR-family).

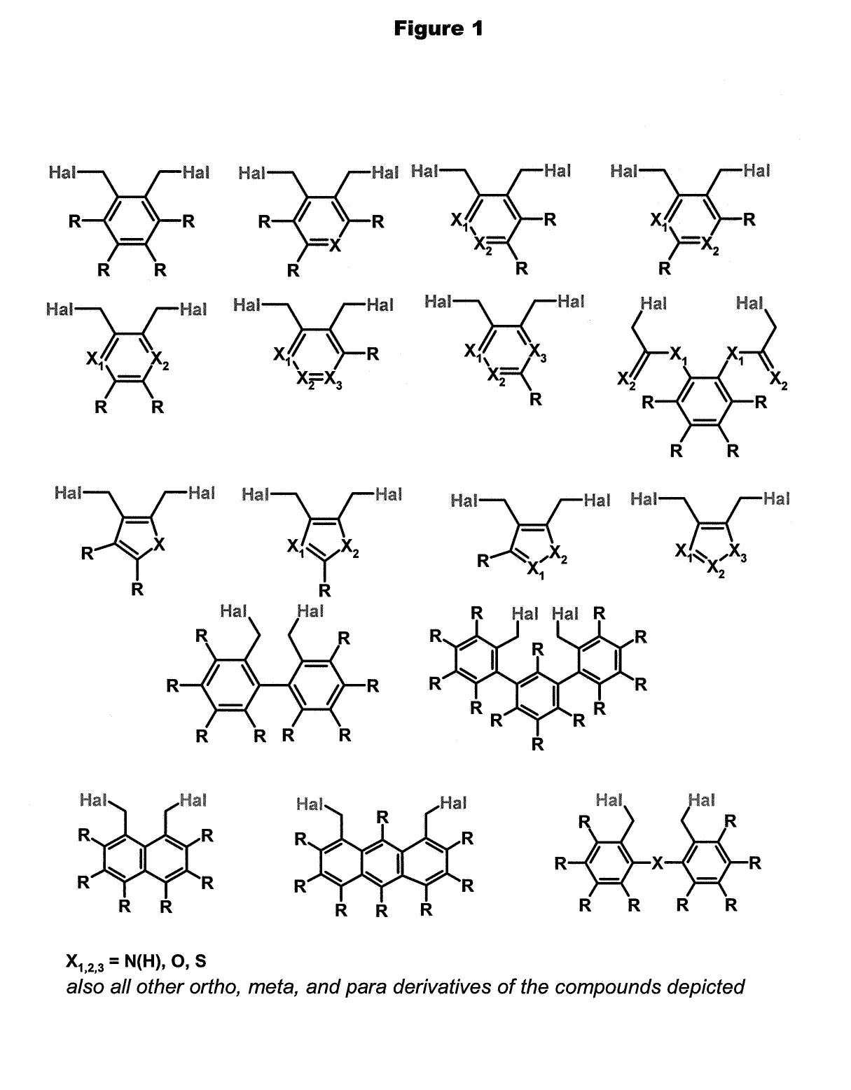

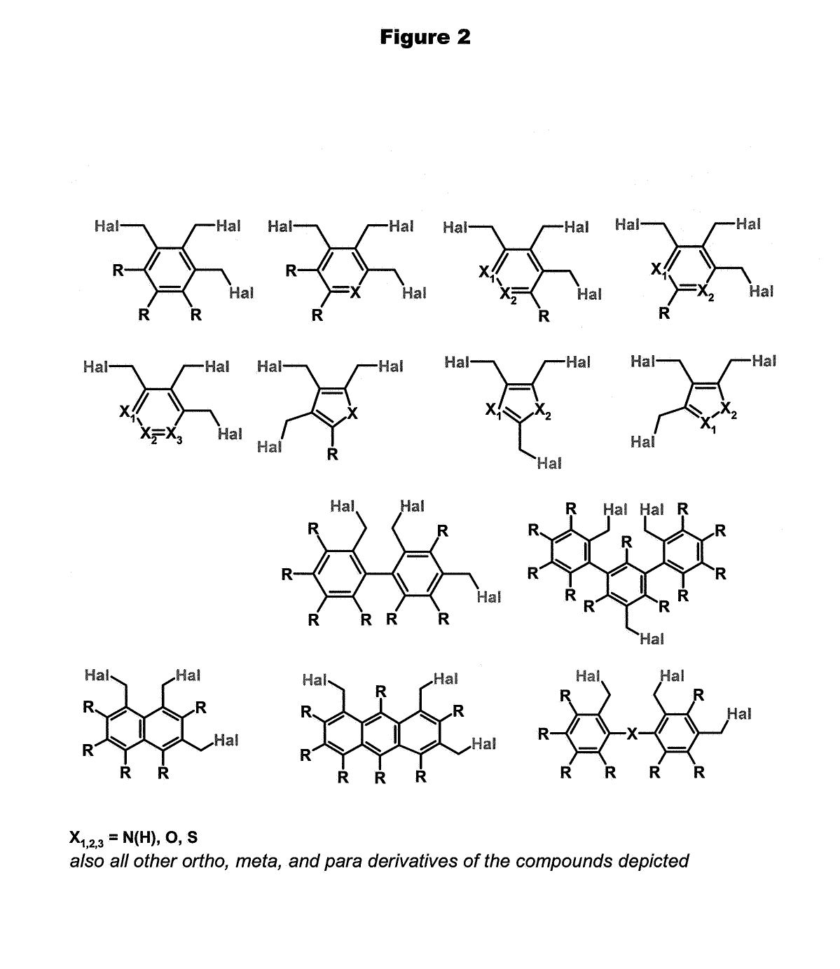

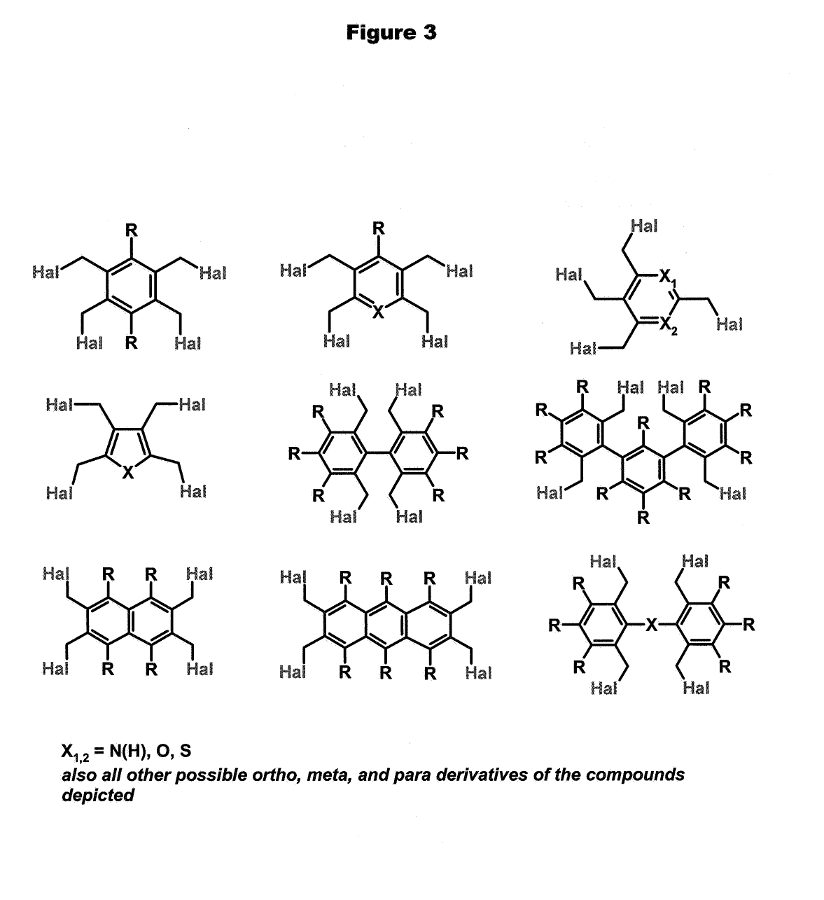

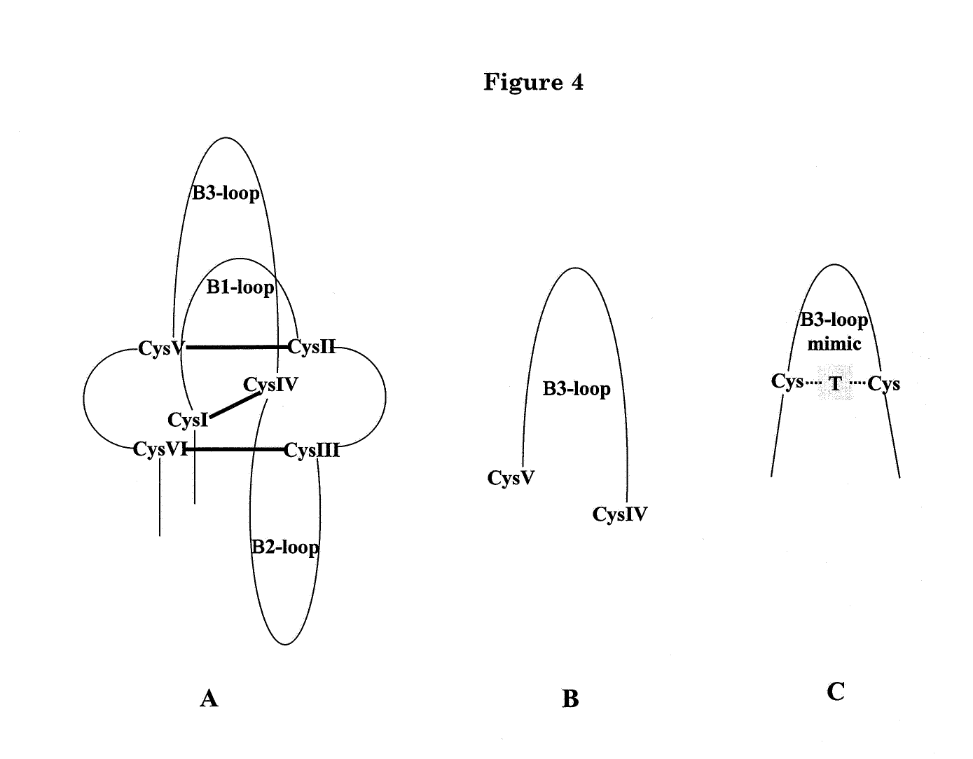

### EXAMPLES

Materials & Methods

Bulk Synthesis of Peptides

Peptides were synthesized by solid-phase peptide synthesis using a 4-(2′,4′-dimethoxyphenyl-Fmoc-aminomethyl)-phenoxy (RinkAmide) resin (BACHEM, Germany) on a Syro-synthesizer (MultiSynTech, Germany). All Fmoc-amino acids were purchased from Orpegen Pharma (Heidelberg, Germany) or Senn Chemicals (Dielsdorf, Switzerland) with side-chain functionalities protected as N-t-Boc (KW), O-t-Bu (DESTY), N-Trt (HNQ), S—Trt (C), S(Acm) (C), or N-Pbf (R) groups. A coupling protocol using a 6.5-fold excess of HBTU/HOBt/amino acid/DIPEA (1:1:1:2) in NMP with a 30 minute activation time using double couplings was employed. Acetylation of peptides was performed by reacting the resin with NMP/Ac₂O/DIEA (10:1:0.1, v/v/v) for 30 min at room temperature. Acylated peptides were cleaved from the resin by reaction with TFA (15 ml/g resin) containing 13.3% (w) phenol, 5% (v) thioanisole, 2.5% (v) 1,2-ethanedithiol, and 5% (v) milliQ-H₂O for 2-4 hrs at RT. The crude peptides were purified by reversed-phase high performance liquid chromatography (RP-HPLC), either on a “DeltaPack” (25×100 or 40×210 mm inner diameter, 15 um particle size, 100 A pore size; Waters, USA) or on a “XTERRA” (19×100 mm inner diameter, 5 um particle size (Waters, USA) RP-18 preparative C₁₈ column with a lineair AB gradient of 1-2% B/min. where solvent A was 0.05% TFA in water and solvent B was 0.05% TFA in ACN. The correct primary ion molecular weights of the peptides was confirmed by electron-spray ionization mass spectrometry on a Micromass ZQ (Micromass, The Netherlands) or a VG Quattro II (VG Organic, UK) mass spectrometer.

Amino acids are indicated by the single-letter code. The asterisk (*) indicates acetylation of the N-terminus and the number sign (#) amidation of the C-terminus.

Synthesis of mT2-Peptides

Peptides containing two free cysteines (C_{T}) were cyclized onto a mT2 CLIPS via reaction with 1.05 equivalent of 1,3-(bisbromomethyl)benzene in 25% ACN/75% ammonium bicarbonate (20 mM, pH 7.8) for 3 hours at room temperature. The T2-peptide constructs was purified by RP-HPLC, followed by treatment with an anion-exchange resin for 2-3 h at room temperature. Finally, the peptide-construct was freeze-dried (3×) from ACN/milliQ-H₂O solution in order to ensure complete removal of traces of TFA and/or ammonium bicarbonate. 1,3-(bisbromomethyl)benzene (mT2) was purchased from Sigma-Aldrich (Zwijndrecht, The Netherlands).

Synthesis of T2/SS or P2/SS-Peptides

Peptides, containing two free cysteines (C_{T}) and two Acm-protected cysteines (C_{S}), were cyclized onto a T2/P2 CLIPS via reaction with 1.05 equivalent of the corresponding (bisbromomethyl)benzene or pyridine compound in 25% ACN/75% ammonium bicarbonate (20 mM, pH 7.8) for 3 hours at room temperature. For deprotection of the Acm-groups and subsequent SS-oxidation, the T2/P2-peptide constructs were treated with excess (10 equiv.) of 12 in a mixture of MeOH/DMSO (9:1, v/v) at 1 mM (final concentration) for 15 mM. at room temperature, followed by destruction of excess of I₂ with vitC. The reaction mixtures were then diluted with 9 volumes of H₂O and filtered over a RP C₁₈-cartridge (Sep-Pak® Vac 3 cc for HPLC-extraction, Waters Corporation, Mass., USA). The peptide-constructs were then collected by elution with ACN/H₂O (6 mL, 1:1 v/v) followed by removal of the solvent by freeze-drying. The peptide-constructs were further purified by RP-HPLC, followed by treatment with an anion-exchange resin (BIO-RAD, AG 1-X8 resin, 100-200 mesh) for 2-3 h at room temperature. Finally, the peptide-constructs were freeze-dried (3×) from ACN/milliQ-H₂O solution in order to ensure complete removal of traces of TFA and/or ammonium bicarbonate.

1,2-(oT2), 1,3-(mT2), 1,4-(bisbromomethyl)benzene (pT2), and 1,3-bis(bromomethyl)pyridine (mP2) were purchased from Sigma-Aldrich (Zwijndrecht, The Netherlands).

Vaccination Experiments

Male Wistar rats were immunized on day 0 with 400 uL of a ˜2.5 mg/mL solution of the CLIPS/SS-peptide construct in PBS/CFA 1:1 (v/v) (PBS=Phosphate-Buffered Saline, CFA=Complete Freund's Adjuvance), followed by a booster (same quantity and concentration) at 4 weeks. The rats were bleeded after 8-9 weeks and the antisera collected.

Evaluation of Anti-FSH Peptide Antisera by ELISA

Antisera were analyzed in a FSH-binding ELISA (Greiner, PS; GDA-coating with 1 μg/mL of protein) using 2,2′-azine-di(ethylbenzthiazoline sulfonate) (ABTS) in combination with a peroxidase-labeled Goat-anti-rat serum as second antibody. Commercially available anti-protein antibodies were included in the analysis as positive controls. FSH was purchased from Biotrend.

FSH-Inhibiting Activity of Anti-FSH Peptide Antisera In Vitro.

The FSH-inhibiting activity of the anti-FSH anti-sera was measured in a human FSHR cell-line assay, using Y1 mouse adrenal cells, stably transfected with the cDNA for the hFSHR as described by Westhoff et al. (Biol. Reprod. 1997, 56, 460-468). This assay measures the ability of anti-FSH antibodies to neutralize the hormonal (bio)activity of native FSH onto its cell-surface receptor (FSHR).

Commercially available anti-FSH mAb 6602 was used as positive control. FSH was purchased from Biotrend.

Results

### Example 1

Effect of Presence of C_{T}C-(61-74) and C_{SS}C-constraint (56-79) on the Ability of Peptides to Generate hFSH-Crossreactive Antisera.

The following hFSH-derived peptides were synthesized and their ability to raise hFSH cross-reactive antibodies was assessed:

1. single-constrained peptide (C_{T}C alone)

2-5. double-constrained peptide (C_{T}C fixed at 61-74; C_{S}C at 4 different positions).

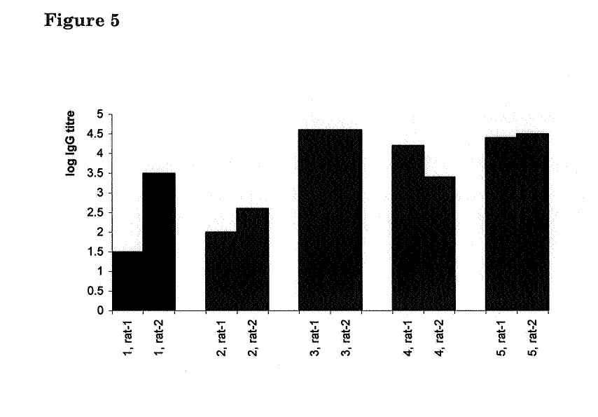

The results are shown in Table 3 and FIG. 5.

This experiment shows that doubly-constrained FSH protein β3-loop mimics that are conformationally rigified both via an mT2-CLIPS at position 61 and 74 plus an internal disulfide (SS) bond between the positions 55 and 80 or 57 and 78, give higher and more reproducible anti-FSH antibody (and neutralization) titres (2×4.6 and 4.5/4.4 for peptides 3 and 5). The position of the internal disulfide (SS) bond was systematically varied in this experiment and found to be optimal for 55-80 and 57-78. The lack of anti-FSH neutralizing for peptide 2 is due to a partial deletion of the binding epitope (V₅₇ and V₇₈) that is necessary for generating the correct antibodies.

The anti-FSH antibody titres obtained with a method according to the invention are significantly higher and:more reproducible (2×4.6 i.s.o. 3.5/1.5) than for a corresponding singly-constrained peptide rigidified only via an mT2-CLIPS at position 61 and 74 and lacking an internal disulfide (SS) bond (compound 1).

The results clearly demonstrate that doubly-constrained (mT2-CLIPS+SS-bond) FSH β3-loop mimics give much higher anti-FSH Ab-titers than the corresponding singly-constrained compound (compound 1: mT2-CLIPS alone). Moreover, it is shown in FIG. 5 that the antibody titers of two individual rats immunized with the same compound according to the invention (either compound 2, 3, 4 or 5) more closely resemble each other as compared to the antibody titers of two individual rats immunized with the control compound (compound 1). Hence, animal to animal variation is reduced, meaning that immunogenic reproducibility is enhanced.

### Example 2

Ability of Double-Constrained Peptide 3 to Generate hFSH-Crossreactive Antisera and Comparison with Single-Constrained Peptide 7-8 and Lineair Peptide 6.

The following set of hFSH-derived peptides were synthesized and their ability to raise hFSH cross-reactive antibodies was assessed:

A

6. lineair peptide (no constraints)

7. single-constrained peptide (C_{T}C alone)

8. single-constrained peptide (C_{S}C alone)

3 (see Example 1). double-constrained peptide (C_{T}C and C_{S}C).

B

9. lineair peptide (no constraints)

10. single-constrained peptide (C_{T}C alone)

11. single-constrained peptide (C_{S}C alone)

12. double-constrained peptide (C_{T}C and C_{S}C).

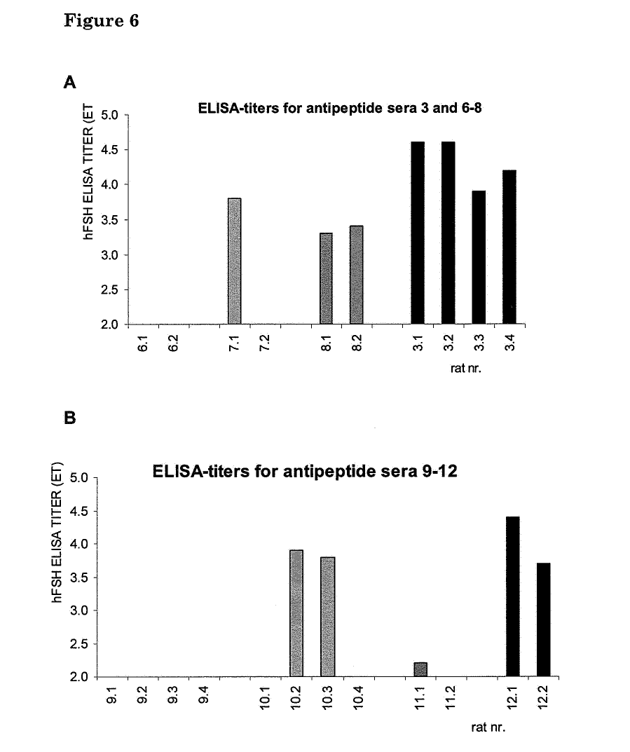

Table 4 and FIG. 6 summarize data on neutralizing activity (NT) and ELISA-titers (ET) for hFSH of rat antisera raised against these 2 sets of peptides, that mimic the beta3-loop of FSH.

This double set of experiments (A and B) clearly show that the ability of doubly-constrained FSH protein β3-loop mimics 3 and 12, conformationally rigified both via an mT2-CLIPS at position 61 and 74 plus an internal disulfide (SS) bond between the positions 56 and 79 is partly lost for the corresponding singly-constrained peptide 7-8 and 10-11, or lost completely for the corresponding lineair peptides 6 and 9. These experiments thus underline the importance of the presence of both constraints. Furthermore, there is a strong correlation between measured ET and NT: High ET's predict high NT's.

Conclusion:

Immunization of rats with peptides (derived from the beta3-loop of FSH) with both a C_{T}C- and C_{S}C-constraint generate reproducibly antisera that strongly bind to hFSH in ELISA and neutralize the bioactivity of hFSH in an FSH-stimulation assay. Lineair peptides totally fail and single-constrained peptides (C_{T}C- or C_{S}C-constraint alone) perform much less good (1-out of-2 response, much lower ET/NT).

### Example 3

Effect of Type of C_{T}C-Constraint (ortho/meta/para-xylyl; 2,6-dimethylpyridyl) on the Ability of Peptides to Generate hFSH-Crossreactive Antisera.

The following set of doubly-constrained hFSH-derived peptides varying in the type of C_{T}C-constraint used to rigidify the peptide's conformation was synthesized and the ability to raise hFSH cross-reactive antibodies was assessed:

3. meta-xylyl linker

13. ortho-xylyl linker

14. para-xylyl linker

15. meta-(2,6-dimethylpyridyl) linker

Table 5 summarizes data on neutralizing activity (NT) and ELISA-titers (ET) for hFSH of rat antisera raised against constrained peptides that mimic the FSH beta3-loop.

These data clearly show that the success of antibody generation as described in Example 1 is almost not dependent on the type of C_{T}C-constraint (ortho/meta/para-xylyl; 2,6-dimethylpyridyl) used to fix the peptides conformation. Results are optimal with a meta-oriented xylyl or dimethylpyridyl-based linker at position 61-74, but results with the ortho/para-xylyl-based linkers are still much better (higher hFSH-ET/NT) than with lineair or single-constrained peptides.

Conclusion: Type of C_{T}C-constraint may vary and is not crucial for the success of antibody generation.

### Example 4A

Effect of Position of C_{T}C-Constraint on Peptides Ability to Generate hFSH-Crossreactive Antisera.

The following set of doubly-constrained hFSH-derived peptides varying in the position of the C_{T}C-constraint was synthesized and the ability to raise hFSH cross-reactive antibodies was assessed:

6,16-19 C_{T}C-constraint “inside” epitope (59-76 to 63-72)

20-22 C_{T}C-constraint “outside” epitope (57-78 to 55-80)

Table 6 summarizes data on neutralizing activity (NT) and ELISA-titers (ET) for hFSH of rat antisera raised against doubly T2/SS-constrained peptides that mimic the FSH beta3-loop.

The data in Table 6 show clearly that placement of the C_{T}C-constraint “inside” epitope (63-72 until 59-76) yields high ET's only for distinct positions (61-74, 63-72), while for the remaining positions (62-73, 60-75, 59-76) the ability to generate hFSH cross-reactive antibodies is totally lost. This is due to removal of crucial amino acids (E₅₉, T₆₀, R₆₂, L₇₃) for antibody recognition of hFSH. Instead, placement of the C_{T}C-constraint “outside” the epitope (57-78 to 55-80) gives high ET's on all positions, indicating that in this case the presence of the C_{T}C-constraint is much less invasive for the peptide's ability to generate the correct hFSH-crossreactive antibodies.

Conclusion: Crucial amino acids should not be used for forming a linkage. A linkage is preferably formed outside an epitope, although non-essential amino acids inside an epitope can also be used.

### Example 4B

Effect of Position of C_{SS}C-Constraint on Peptides Ability to Generate hFSH-Crossreactive Antisera.

The following set of doubly-constrained hFSH-derived peptides varying in the position of the C_{SS}C-constraint was synthesized and the ability to raise hFSH cross-reactive antibodies was assessed:

23 C_{SS}C-constraint at 57-78

24 C_{SS}C-constraint at 55-80

25 C_{SS}C-constraint at 54-81

26 C_{SS}C-constraint at 51-84

27 C_{SS}C-constraint at 48-87

28 C_{SS}C-constraint at 45-90

In addition to this, the following set of singly-constrained peptides lacking the C_{T}C-constraint at position 61-74 was synthesized and studied for comparison:

26-SS only C_{SS}C-constraint at 51-84

27-SS only C_{SS}C-constraint at 48-87

28-SS only C_{SS}C-constraint at 45-90

Table 7 summarizes data on neutralizing activity (NT) and ELISA-titers (ET) for hFSH of rat antisera raised against this set of constrained peptides that mimic the FSH beta3-loop.

Conclusion:

The data show clearly that varying the position of the C_{SS}C-constraint “outside” the epitope (58-79 to 45-90) does not influence much the immunogenic behaviour of the peptides and generates high ET's for all doubly-constrained peptides. In contrast to this, the corresponding singly-constrained peptides with only a C_{SS}C-constraint (26SS-28SS) are much less effective and almost completely fail to produce significant amounts of hFSH-crossreactive antibodies.

### Example 5A

Ability of Double-Constrained Peptides (C_{T}C and C_{S}C) Derived from the Beta5-Loop-Beta6 Region of hVEGF (Yet Another Member of the Cys-Knot Growth Factor Family) to Generate hVEGF-Crossreactive Antisera.

The following set of peptides derived from hVEGF was synthesized and the ability to raise hVEGF cross-reactive antibodies was assessed:

29 lineair peptide (no constraints)

30 single-constrained peptide (C_{T}C at 78-94 alone)

31 double-constrained peptide (C_{T}C at 78-94 and C_{SS}C at 74-98)

The results are shown in Table 8.

Like observed for FSH (see Example 1-4) this experiment again shows that a doubly-constrained peptide derived from the beta5-loop-beta6 region of hVEGF conformationally fixed both via a C_{T}C-constraint at position 78-94 plus C_{SS}C-constraint at positions 74-98, gives higher and more reproducible anti-VEGF antibody (and neutralization) titres (2x>4.4 for peptide 31).

### Example 5B

Ability of Double-Constrained Peptides (C_{T}C and C_{S}C) Derived from the Beta5-Loop-Beta6 Region of hVEGF (Yet Another Member of the Cys-Knot Growth Factor Family) to Block the Binding of Anti-VEGF mAb to Surface-Immobilized hVEGF.

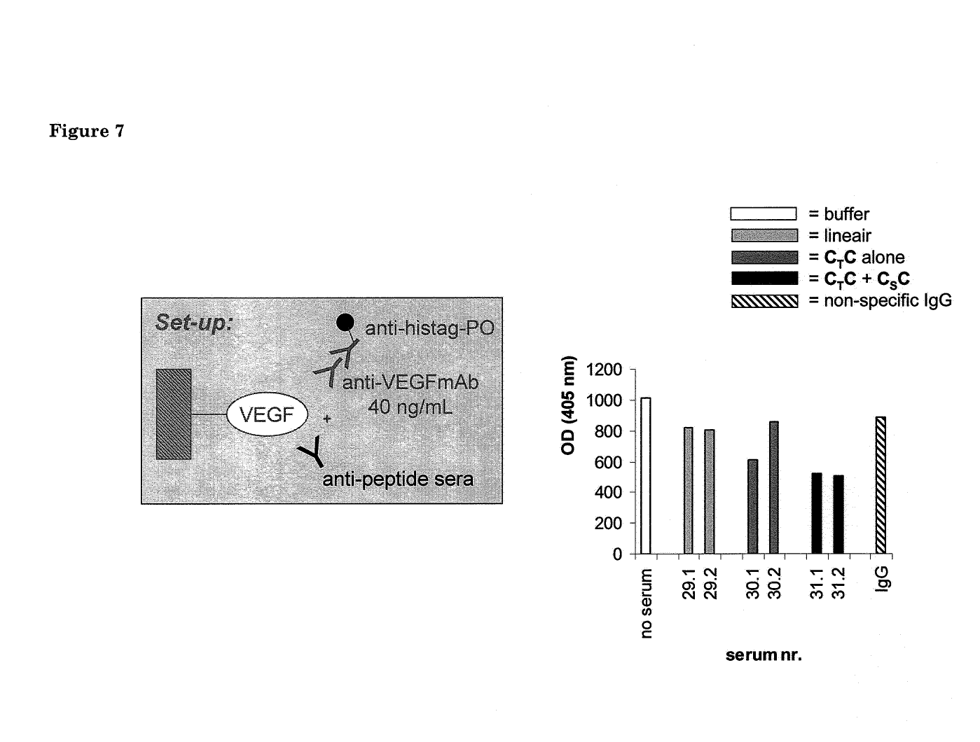

A binding competition experiment was carried out with the 6 antipeptide antisera 29.1-31.2 (see example 5B) in order to assess the ability of the sera to bind to hVEGF. The experimental setup of this experiment is schematically shown in FIG. 7 and is as follows:

ELISA-plates were coated with hVEGF (Greiner, PS; GDA-activation for 3 h in acetate-buffer, pH=5 followed by exposure to hVEGF at 1 μg/mL in phosphate-buffer, pH=8, overnight). Then, the wells were incubated for 1 h at 37 C with 100 uL of a 1:1 mixture of an anti-VEGF mAb (40 ng/mL) and 1 of the 6 antisera (at 1/10 dilution) in 5% horse-serum. The amount of anti-VEGF mAb bound to the surface-immobilized VEGF was then determined using 2,2′-azine-di(ethylbenzthiazoline sulfonate) (ABTS) in combination with a peroxidase-labeled Goat-anti-human serum as second antibody, and used as a measure for the ability of the antipeptide sera to compete for binding with the anti-VEGF mAb. Binding of anti-peptide sera to bind to VEGF and consequently the ability to block mAb-binding translates into low binding levels for the mAb.

The results (see FIG. 7) also show clearly that the performance of anti-peptide sera derived from the doubly-constrained peptide 31 is much stronger than for the corresponding lineair peptide (29) and singly-constrained peptide (30). The ability of the anti peptide sera 29.1/2 to block VEGF-binding of an anti-VEGF mAb is not measurable (equal to that of non-specific IgG), while only ½ sera against the singly-constrained peptide 30 shows activity.

Overall Conclusion:

Immunization of rats with double-constraint peptides as described in Example 1 for hFSH gives very similar results for hVEGF, yet another member of the cys-knot protein-family. The binding of both antisera (2-o-2) generated with double-constrained peptides (both C_{T}C- and C_{S}C-constraint) to hVEGF in ELISA is superior to that of antisera generated with either the lineair (no C_{T}C- and C_{S}C-constraint) or single-constraint (only C_{T}C-constraint) peptides. Moreover, competition studies in. ELISA show clearly that both sera block the binding of an anti-VEGF mAb with surface-immobilized VEGF much more efficiently than the sera of lineair or single-constrained peptides derived from the beta5-loop-beta6 part of hVEGF.

### Example 6

Ability of Double-Constrained Peptides (C_{T}C and C_{S}C) Derived from the Dimerization Arm in Domain-II (CR1) of the ErbB2 (HER2/neu) Protein Belonging to the Epidermal Growth Factor Receptor Family (EGFR) to Generate ERbB2-Crossreactive Antisera.

The following set of peptides derived from ErbB2 (HER2/neu) was synthesized and the ability to raise cross-reactive antibodies to ErbB2 was assessed:

32. lineair peptide (no constraints)

33. single-constrained peptide (C_{T}C at 246-266 alone)

34 double-constrained peptide (C_{T}C at 251-260 and C_{SS}C at 246-266).

35 double-constrained peptide (C_{T}C at 254-256 and C_{SS}C at 246-266).

Conclusion:

The data in Table 9 clearly show that immunization of rats with double-constraint peptides as described in Example 1 for hFSH gives very similar results for double-constrained peptides derived from the dimerization arm of domain-II (CR1) of the ERbB2 (HER2/neu) receptor protein, a member of the EGFR-family of receptor proteins. In total 4 antisera (2 out of 2) generated with 2 different double-constrained peptides (both C_{T}C- and C_{S}C-constraint) bind strongly to the ERbB2 protein in ELISA. In contrast to this, the antisera obtained with the corresponding lineair peptide bind only weakly (1-o-2; ET=2.4; <2.0) to ERbB2, whereas antisera obtained with a single-constrained peptide (only C_{T}C-constraint) none of the two sera binds to the ERbB2 protein in ELISA (ET=<2.0).

### Example 7

Ability of Double-Constrained Peptides (C_{T}C and C_{S}C) Derived from the Extracellular Domain Loop-2 (ECL-2) of the CCR5-Receptor Protein Belonging to the Family of G-Protein Coupled Receptors (GPCR's) to Mimic Letter the Receptor Protein.

The following set of peptides derived from the ECL-2 loop of CCR5 was synthesized and studied for the ability to mimic the receptor protein in binding to an anti-CCR5 monoclonal antibody:

36. lineair peptide (no constraints)

37. single-constrained peptide (C_{T}C at 169-178 alone)

38 double-constrained peptide (C_{T}C at 169-178 and C_{SS}C at 167-180).

39 double-constrained peptide (C_{T}C at 169-178 and C_{SS}C at 166-181).

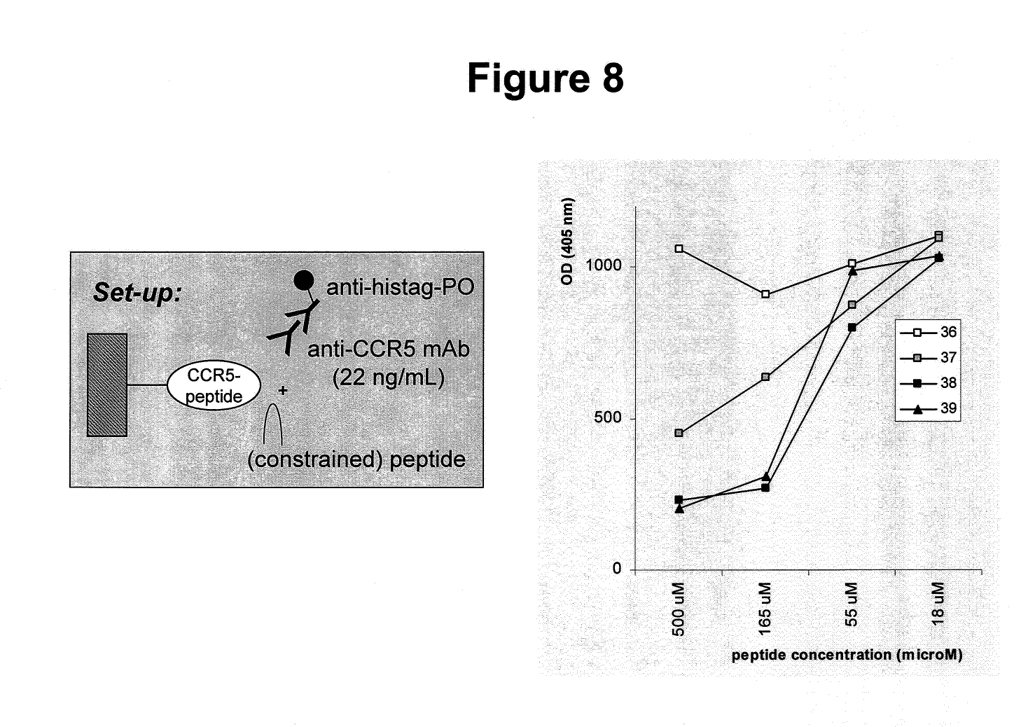

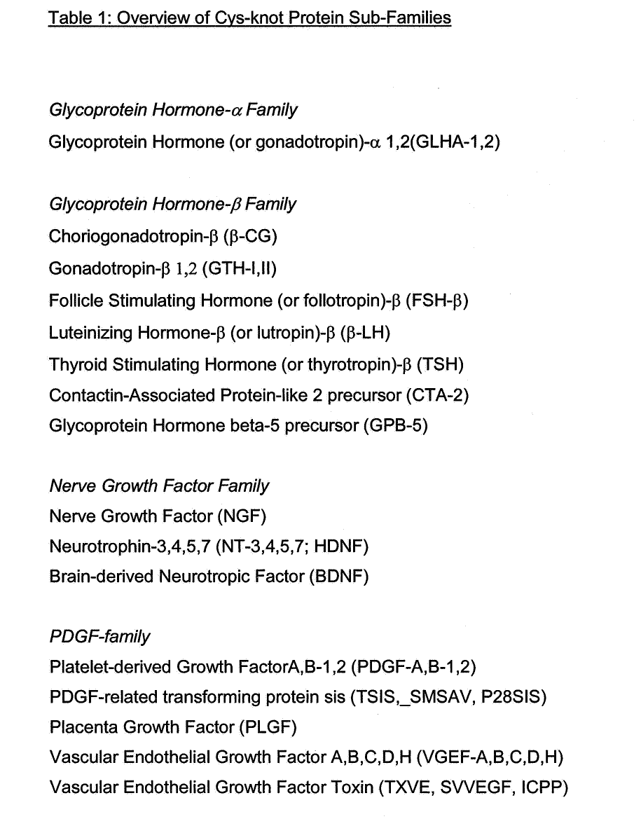

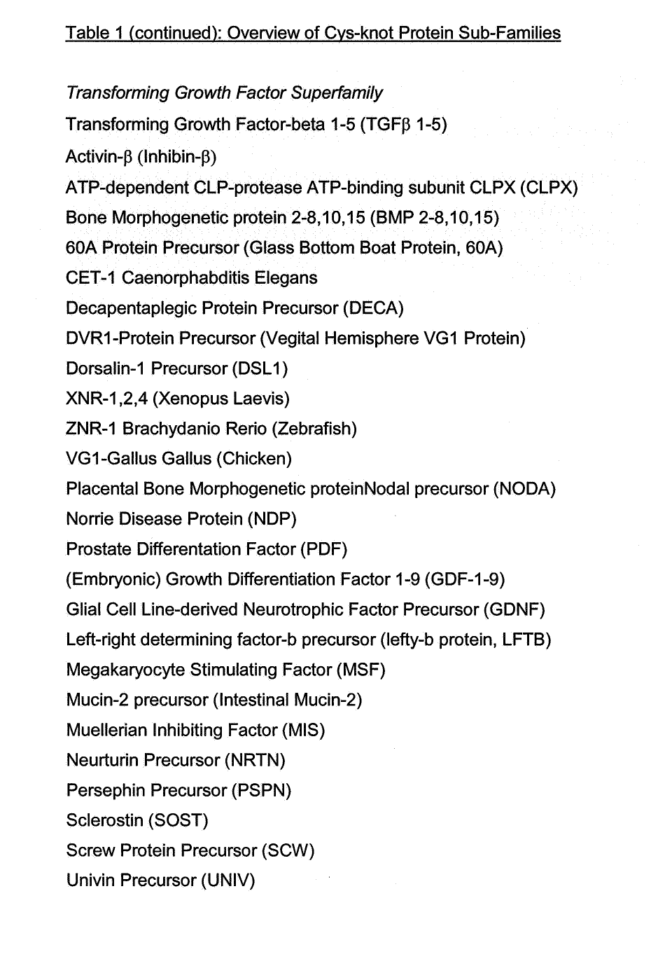

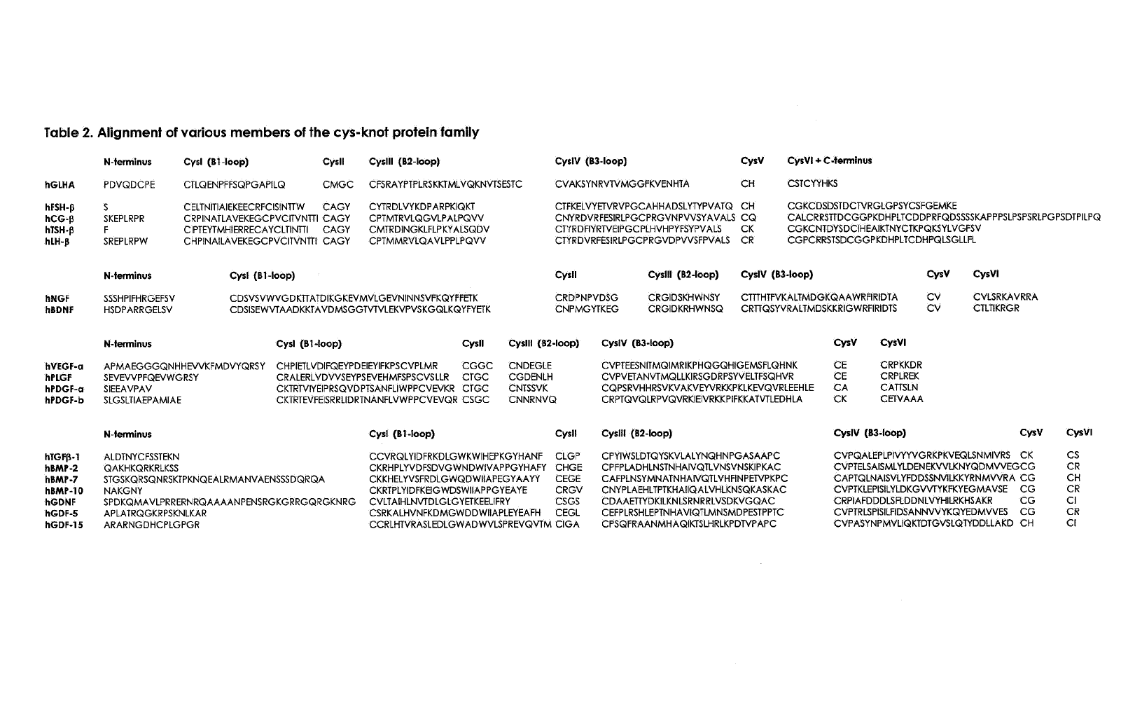

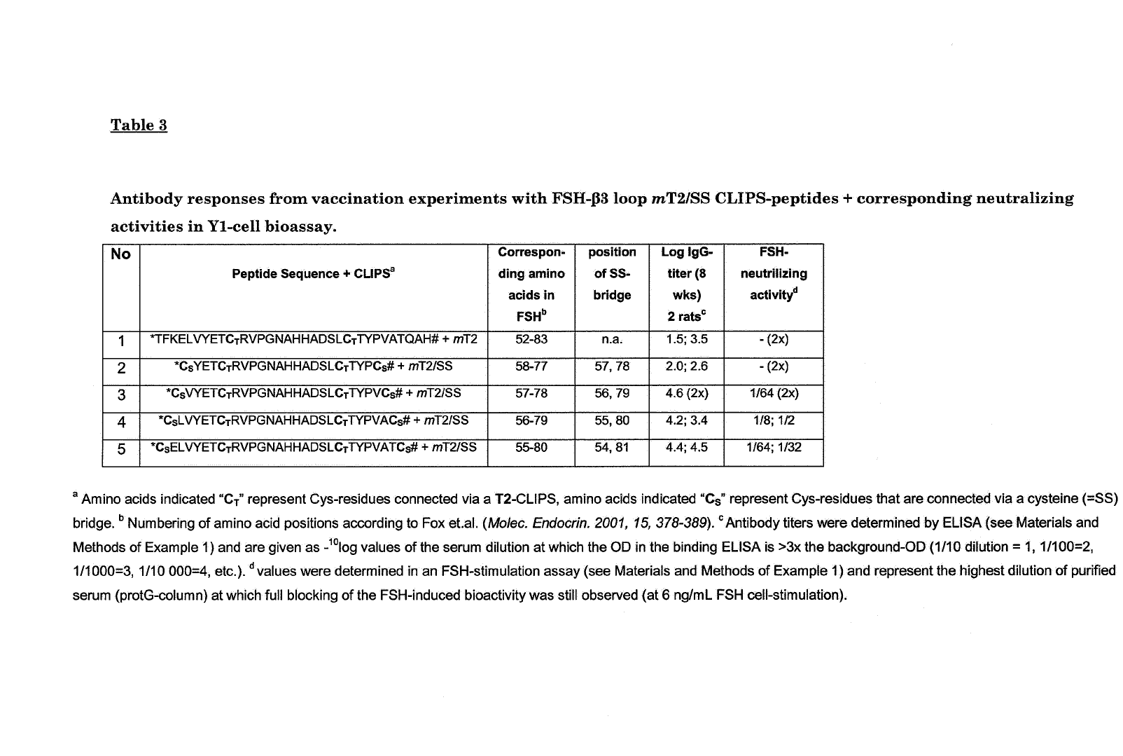

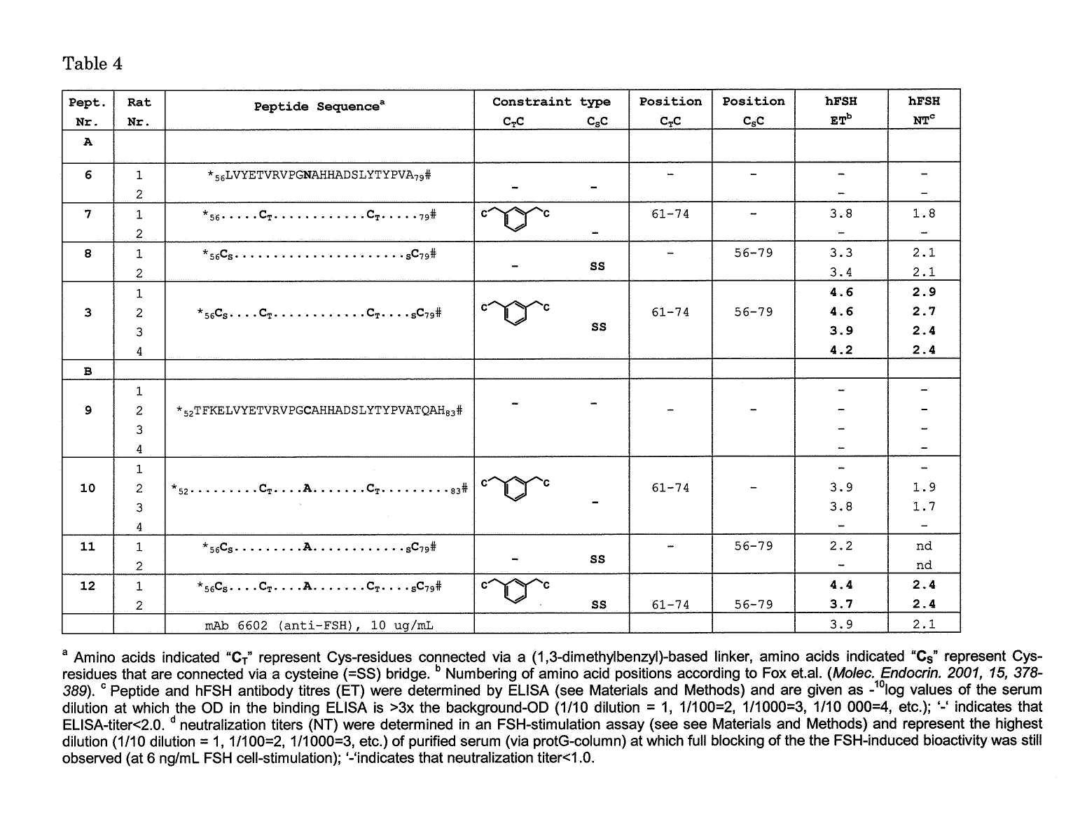

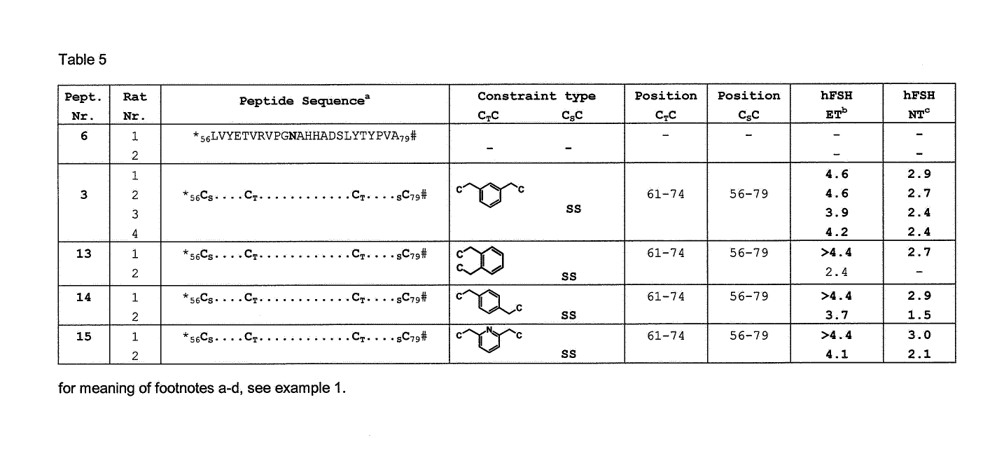

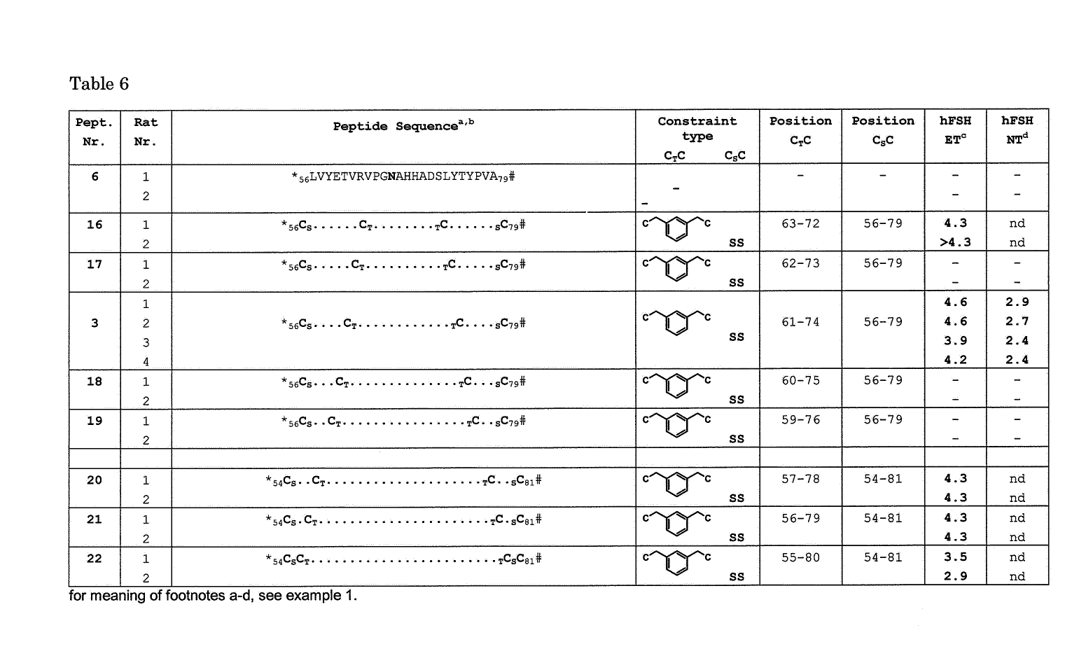

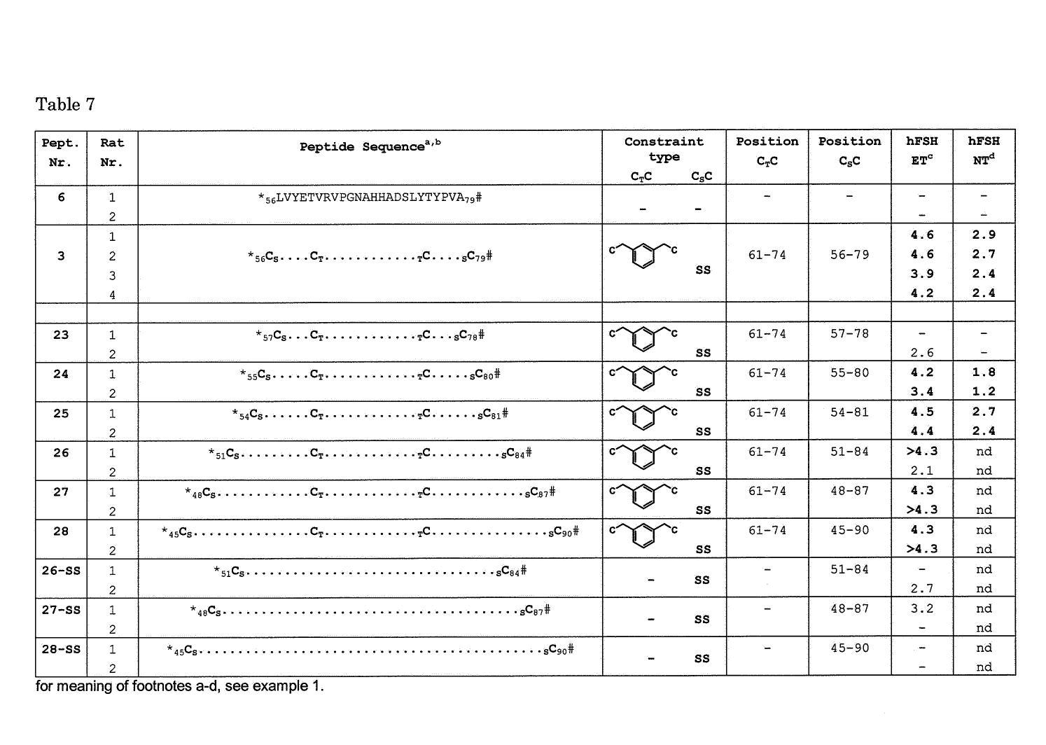

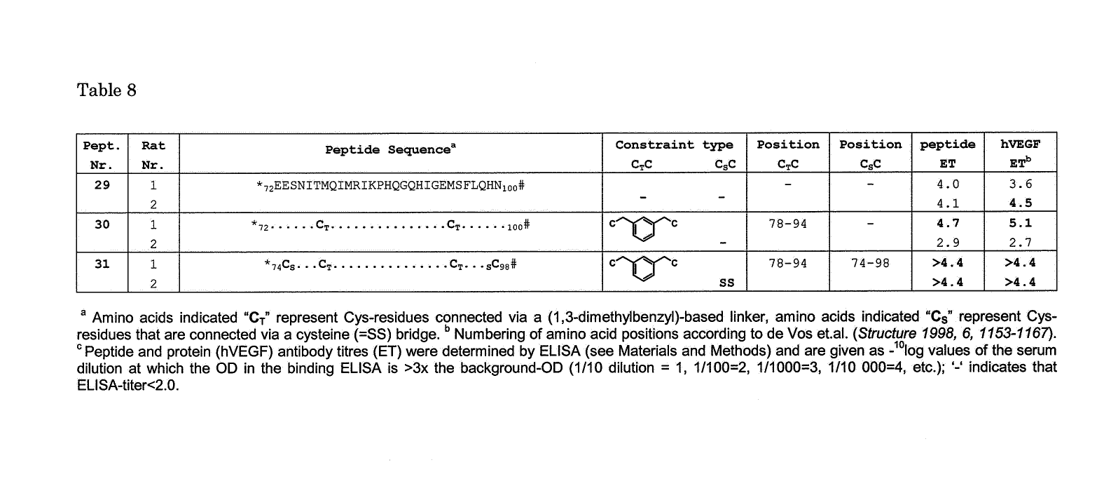

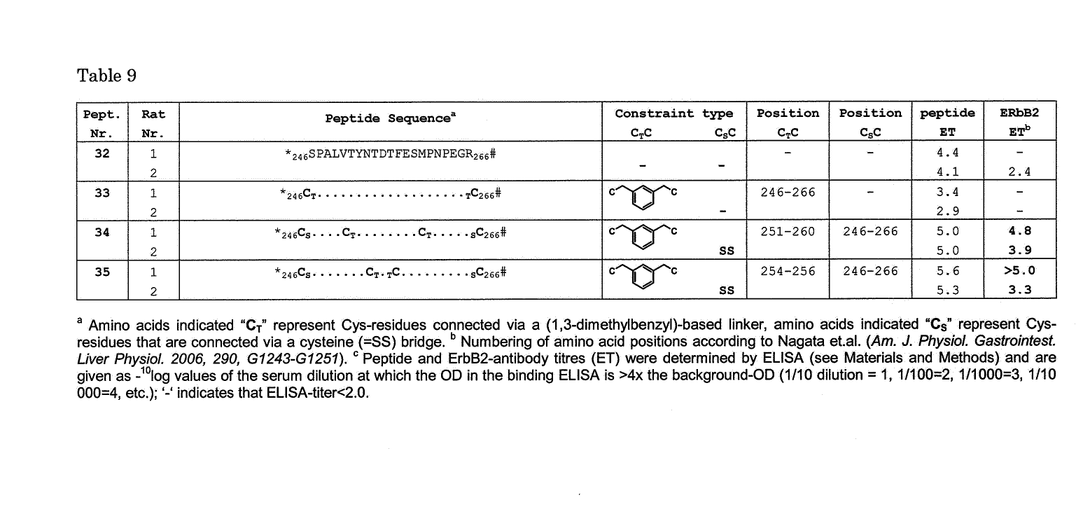

A binding competition experiment was carried out with these 4 peptides in order to evaluate their ability to bind to the anti-CCR5 monoclonal antibody 2D7 (see also FIG. 8). This provides a means to determine their potential to functionally mimic the surface of the CCR5-receptor protein and so be a good immunogen for generating antibodies against CCR5 (immunogenicity for FSH-derived peptides as shown in example 1-4 exactly parallels the improved ability of these peptides to block the binding of anti-FSH mAb's in an ELISA-competition assay (data not shown)). The ability of peptides to bind to mAb 2D7 was measured in ELISA, with a peptide derived from the ECL2a-loop of CCR5 immobilized to the surface. mAb 2D7 shows strong binding to this peptide in ELISA. Binding of peptides 36-39 to 2D7 in solution can now be studied in competition with binding to the surface-immobilized peptide, and translates into decreased surface binding of mAb 2D7.

The experimental setup of the competition experiment is schematically shown in FIG. 8 and goes as follows: ELISA-plates were coated with double-constrained peptide *C_{S}FTRC_{T}TQKEGLHYTC_{T}TSSHC_{S}# (Greiner, PS; GDA-activation for 3 h in acetate-buffer, pH=5 followed by exposure to constrained peptide at 10 microgram/mL in phosphate-buffer, pH=8, overnight). Then, the wells were incubated for 1 h at 37 C with 100 uL of a 1:1 mixture of mAb 2D7 (22 ng/mL) and 1 of the 4 peptides (start concentration 500 microM; subsequent steps of ⅓ dilution) in 5% horse-serum. The amount of mAb 2D7 bound to the surface-immobilized constrained peptide was then determined using 2,2′-azine-di(ethylbenzthiazoline sulfonate) (ABTS) in combination with a peroxidase-labeled Rabbit-anti-mouse serum as second antibody.

The results (see FIG. 8) show clearly that the performance of double-constrained peptides (38-39) is much stronger than of the corresponding lineair (36) and single-constrained peptide (37). The ability of lineair peptide 36 to block 2D7-binding is not detectable, even not at the highest possible concentration, while that for single-constrained peptide 37 is ˜50% at 500 micromolar. However, for the double-constrained peptides 38 and 39, the effectiveness is much higher as they even inhibit 2D7-binding for ˜90% at 165 microM concentration. This clearly illustrates the improved potential of double-constrained peptides in mAb-binding peptides.

Conclusion:

Double-constrained peptides derived from the ECL2-loop of receptor protein CCR5 provide much better mimics of this receptor than the corresponding lineair or single-constrained peptides, as established in a competition ELISA experiments. This will undoubtedly translate into improved ability of these peptides to generate antibodies or antisera that are cross-reactive with native CCR5 upon immunization.

1. A method for inducing and/or enhancing immunogenic reproducibility and/or immunogenicity of a compound, the method comprising at least in part restricting the conformation of said compound by the formation of a linkage at at least two different sites of said compound, wherein said compound is attached to at least one scaffold and/or carrier and by the formation of at least one internal bond within said compound.

2.-4. (canceled)

5. The method according to claim 1, wherein said compound is attached to said scaffold and/or carrier via at least two linkages.

6. The method according to claim 1, wherein said scaffold comprises:
a halogenoalkane, preferably a dihaloalkane, a trihaloalkane or a tetrahaloalkane; and/or
an allylic system, preferably a scaffold comprising two allylic halogen atoms; and/or
a scaffold comprising at least two halomethyl groups; and/or
a (hetero)aromatic molecule, preferably a (hetero)aromatic molecule comprising at least two benzylic halogen substitutents.

7. The method according to claim 1, wherein said scaffold is a halomethylarene, preferably selected from the group consisting of bis(bromomethyl)benzene, tris(bromomethyl)benzene and tetra(bromomethyl)benzene, or a derivative thereof.

8.-10. (canceled)

11. A method for inducing and/or enhancing immunogenic reproducibility and/or immunogenicity of a compound, the method comprising at least in part restricting the conformation of said compound by the formation of at least two internal bonds within said compound.

12. The method according to claim 1, wherein said compound is attached to a carrier.

13. An immunogenic compound comprising an amino acid sequence bound to a scaffold and/or a carrier, wherein said amino acid sequence comprises at least one internal bond.

14. (canceled)

15. The immunogenic compound according to claim 13, wherein said amino acid sequence is attached to said scaffold via at least two linkages.

16. The immunogenic compound according to claim 13, wherein said scaffold comprises a (hetero)aromatic molecule, preferably a (hetero)aromatic molecule comprising at least two benzylic halogen substitutents.

17. The immunogenic compound according to claim 13, wherein said scaffold is a halomethylarene, preferably selected from the group consisting of bis(bromomethyl)benzene, tris(bromomethyl)benzene and tetra(bromomethyl)benzene, or a derivative thereof.

18. (canceled)

19. The immunogenic composition comprising an immunogenic compound according to claim 13 and a pharmaceutically acceptable carrier, diluent and/or excipient.

20.-24. (canceled)

25. A method for preparing the compound of claim 13, the method comprising:
providing a scaffold comprising at least a first and a second reactive group;
providing at least one molecule capable of reacting with said at least first and second reactive group, said molecule comprising an amino acid sequence;
contacting said scaffold with said at least one molecule to form at least two linkages between said scaffold and said at least one molecule in a coupling reaction, whereby the formation of a linkage accelerates the formation of a consecutive linkage, preferably wherein said coupling reaction is performed in solution, more preferably in an aqueous solution; and
allowing, inducing and/or enhancing the formation of an internal bond within said molecule.

26. (canceled)

27. The method according to claim 25, wherein said scaffold comprises a (hetero)aromatic molecule, preferably a (hetero)aromatic molecule comprising at least two benzylic halogen substitutents.

28. The method according to claim 25, wherein said scaffold is a halomethylarene, preferably selected from the group consisting of bis(bromomethyl)benzene, tris(bromomethyl)benzene and tetra(bromomethyl)benzene, or a derivative thereof.

29. (canceled)

30. A method for vaccinating an animal against a tumor-related disease, comprising administering to said animal a suitable dose of the immunogenic compound of claim 13, wherein said immunogenic compound comprises an amino acid sequence comprising a sequence which is at least 50%, preferably at least 60%, more preferably at least 70% homologous to at least part of the amino acid sequence of VEGF, hCG, PlGF, HER and/or HGF/SF, said part having a length of at least 8 amino acid residues.

31. A method for vaccinating an animal against angiogenesis, comprising administering to said animal a suitable dose of the immunogenic compound of claim 13, wherein said immunogenic compound comprises an amino acid sequence comprising a sequence which is at least 50%, preferably at least 60%, more preferably at least 70% homologous to at least part of the amino acid sequence of VEGF, said part having a length of at least 8 amino acid residues.

32. A method for vaccinating an animal against the formation of metastases during a tumor-related disease, preferably prostate cancer, comprising administering to said animal a suitable, dose of the immunogenic compound of claim 13, wherein said immunogenic compound comprises an amino acid sequence comprising a sequence which is at least 50%, preferably at least 60%, more preferably at least 70% homologous to at least part of the amino acid sequence of HGF/SF, said part having a length of at least 8 amino acid residues.

33. A method for producing antibodies, T cells and/or B cells, comprising:
immunizing a non-human animal with the immunogenic compound of claim 13 and/or the immunogenic composition of claim 19, and
harvesting antibodies, T cells and/or B cells capable of specifically binding said immunogenic compound from said animal.

34. The method according to claim 33, further comprising producing monoclonal antibodies using said antibody obtained from said animal.

35. (canceled)

36. A protein mimic comprising an amino acid sequence bound to a scaffold and/or a carrier via at least two linkages, wherein said amino acid sequence comprises at least one internal SS-bridge.
